SEQUENCE LISTING (34 sequences)

SEQ ID NO: 1 - protein, 252 aa, Lactobacillus brevis
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSIEGFVGDPSLGAYNASKGAVRIMSKSAALDCALKDYDVRVNTVHPGYIKTPLVDDLPGAEEAMSQRTKTPMGHIGEPNDIAYICVYLASNESKFATGSEFVVDGGYTAQ

SEQ ID NO: 2 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDLPGLEEAMSQRTKTPMGHIGEPNDIAYICVYLASNESKFATGSEFVVDGGYTAQ

SEQ ID NO: 3 - protein, 252 aa, synthetic construct
MSNRLDGKVAIITGGTLGIGLAIATKFVEEGAKVMITGRHSDQGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDLPGCEEAMSQRTKTPMGHIGEPNDIAYICVYLASNESKFATGSEFVVDGGYTAQ

SEQ ID NO: 4 - protein, 252 aa, synthetic construct
MSNRLDGKVAIITGGTLGIGLAIATKFVEEGAKVMITGRHSDQGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDLPGEEEAMSQRTKTPMGHIGEPNDIAYICVYLASNESKFATGSEFVVDGGYTAQ

SEQ ID NO: 5 - protein, 252 aa, synthetic construct
MSNRLDGKVAIITGGTLGIGLAIATKFVEEGAKVMITGRHSDQGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDLPGIEEAMSQRTKTPMGHIGEPNDIAYICVYLASNESKFATGSEFVVDGGYTAQ

SEQ ID NO: 6 - protein, 252 aa, synthetic construct
MSNRLDGKVAIITGGTLGIGLAIATKFVEEGAKVMITGRHSDQGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDLPGTEEAMSQRTKTPMGHIGEPNDIAYICVYLASNESKFATGSEFVVDGGYTAQ

SEQ ID NO: 7 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDMPGIEEAMSQRTKTPMGHIGEPNDIAYICVYLASNESKFATGSEFVVDGGYTAQ

SEQ ID NO: 8 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDMPGLEEAMSQRTKTPMGHIGEPNDIAYICVYLASNESKFATGSEFVVDGGYTAQ

SEQ ID NO: 9 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDNPGLEEAMSQRTKTPMGHIGEPNDIAYICVYLASNESKFATGSEFVVDGGYTAQ

SEQ ID NO: 10 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDNPGIEEAMSQRTKTPMGHIGEPNDIAYICVYLASNESKFATGSEFVVDGGYTAQ

SEQ ID NO: 11 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDSPGLEEAMSQRTKTPMGHIGEPNDIAYICVYLASNESKFATGSEFVVDGGYTAQ

SEQ ID NO: 12 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSILGFVGDPSLGAYNASKGAVRIMSKSAALDCALKDYDVRVNTVHPGYIKTPLVDDSPGIEEAMSQRTKTPMGHIGEPNDIAYICVYLASNESKFATGSEFVVDGGYTAQ

SEQ ID NO: 13 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSALGFVGDPSLGAYNASKGAVRIMSKSAALDCALKDYDVRVNTVHPGYIKTPLVDDMPGIEEAMSQRTKTPMGHIGEPNDIAYICVYLASNESKFATGSEFVVDGGYTAQ

SEQ ID NO: 14 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINISSILGFVGDPSLGAYNASKGAVRIMSKSAALDCALKDYDVRVNTVHPGYIKTPLVDDSPGIEEAMSQRTKTPMGHIGEPNDIAYICVYLASNESKFATGSEFVVDGGYTAQ

SEQ ID NO: 15 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSALGFVGDPSLGAYNASKGAVRIMSKSAALDCALKDYDVRVNTVHPGYIKTPLVDDNPGIEEAMSQRTKTPMGHIGEPNDIAYICVYLASNESKFATGSEFVVDGGYTAQ

SEQ ID NO: 16 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSALGFVGDPSLGAYNASKGAVRIMSKSAALDCALKDYDVRVNTVHPGYIKTPLVDDMPGLEEAMSQRTKTPMGHIGEPNDIAYICVYLASNESKFATGSEFVVDGGYTAQ

SEQ ID NO: 17 - protein, 252 aa, synthetic construct
MSNRLDGKVAIITGGTLGIGLAIATKFVEEGAKVMITGRHSDVGEKAAKSVGTPDQIQFFQHDSSDEDGWTKLFDATEKAFGPVSTLVNNAGIAVNKSVEETTTAEWRKLLAVNLDGVFFGTRLGIQRMKNKGLGASIINMSSALGFVGDPSLGAYNASKGAVRIMSKSAALDCALKDYDVRVNTVHPGYIKTPLVDDSPGLEEAMSQRTKTPMGHIGEPNDIAYICVYLASNESKFATGSEFVVDGGYTAQ

SEQ ID NO: 18 - DNA, 759 bp, Lactobacillus brevis
  source 1..759: mol_type = genomic DNA
atgagcaaccgtctggacggcaaggtggcgatcattaccggtggcaccctgggtattggtctggcgattgcgaccaagttcgtggaggaaggtgcgaaagttatgatcaccggccgtcacagcgacgtgggcgagaaggcggcgaaaagcgttggcaccccggaccagattcaattctttcagcacgatagcagcgacgaggatggttggaccaagctgttcgatgcgaccgaaaaagcgtttggcccggttagcaccctggttaacaacgcgggtattgcggtgaacaagagcgttgaggaaaccaccaccgcggagtggcgtaaactgctggcggtgaacctggatggtgttttctttggcacccgtctgggtatccaacgtatgaagaacaaaggtctgggcgcgagcatcattaacatgagcagcattgaaggtttcgttggcgacccgagcctgggtgcgtacaacgcgagcaagggtgcggttcgtatcatgagcaaaagcgcggcgctggattgcgcgctgaaggactacgatgtgcgtgttaacaccgtgcacccgggctatattaaaaccccgctggttgacgatctgccgggtgcggaggaagcgatgagccagcgtaccaagaccccgatgggtcacatcggcgaaccgaacgacatcgcgtacatttgcgtttatctggcgagcaacgagagcaaattcgcgaccggtagcgaatttgtggttgatggtggctataccgcgcaataa

SEQ ID NO: 19 - DNA, 759 bp, synthetic construct
  source 1..759: mol_type = other DNA
atgtccaatcgcttggacgggaaggttgcgattattaccggtggcaccctgggcatcggcctggcgatcgctactaaatttgtggaagaaggtgccaaggtcatgattaccggccgtcacagcgatgtaggcgaaaaagcagcaaagtccgtcgggacccctgatcagattcaattctttcaacacgattcgagcgacgaggatggatggactaaattgtttgatgccaccgaaaaggcattcggtcctgtaagtaccttggtcaacaatgcaggcatcgctgtaaacaaaagcgtcgaggagactactacggcagaatggcgcaaacttctggccgtcaacttggacggcgttttttttggcacgcgtctgggcattcaacgtatgaaaaacaaaggtttgggagcgtccatcatcaatatgagcagcatccttggattcgtaggggacccgtcgctgggtgcatacaacgcctcgaaaggggcggtgcgcattatgtcaaaaagcgcggccctggactgtgccttaaaagattatgatgtacgcgtgaacacagttcatcccggttacattaaaaccccgcttgtcgatgatctccccggcctggaggaagcgatgtctcagcgcaccaaaacgccgatgggccacattggcgaacctaacgatatcgcatatatttgcgtttacctggcaagcaatgaatctaaatttgcgaccggctcagagtttgtggtggatggcggctataccgcgcagtaa

SEQ ID NO: 20 - DNA, 759 bp, synthetic construct
  source 1..759: mol_type = other DNA
atgtccaatcgcttggacgggaaggttgcgattattaccggtggcaccctgggcatcggcctggcgatcgctactaaatttgtggaagaaggtgccaaggtcatgattaccggccgtcacagcgatgtaggcgaaaaagcagcaaagtccgtcgggacccctgatcagattcaattctttcaacacgattcgagcgacgaggatggatggactaaattgtttgatgccaccgaaaaggcattcggtcctgtaagtaccttggtcaacaatgcaggcatcgctgtaaacaaaagcgtcgaggagactactacggcagaatggcgcaaacttctggccgtcaacttggacggcgttttttttggcacgcgtctgggcattcaacgtatgaaaaacaaaggtttgggagcgtccatcatcaatatgagcagcatccttggattcgtaggggacccgtcgctgggtgcatacaacgcctcgaaaggggcggtgcgcattatgtcaaaaagcgcggccctggactgtgccttaaaagattatgatgtacgcgtgaacacagttcatcccggttacattaaaaccccgcttgtcgatgatctccccggctgcgaggaagcgatgtctcagcgcaccaaaacgccgatgggccacattggcgaacctaacgatatcgcatatatttgcgtttacctggcaagcaatgaatctaaatttgcgaccggctcagagtttgtggtggatggcggctataccgcgcagtaa

SEQ ID NO: 21 - DNA, 759 bp, synthetic construct
  source 1..759: mol_type = other DNA
atgtccaatcgcttggacgggaaggttgcgattattaccggtggcaccctgggcatcggcctggcgatcgctactaaatttgtggaagaaggtgccaaggtcatgattaccggccgtcacagcgatgtaggcgaaaaagcagcaaagtccgtcgggacccctgatcagattcaattctttcaacacgattcgagcgacgaggatggatggactaaattgtttgatgccaccgaaaaggcattcggtcctgtaagtaccttggtcaacaatgcaggcatcgctgtaaacaaaagcgtcgaggagactactacggcagaatggcgcaaacttctggccgtcaacttggacggcgttttttttggcacgcgtctgggcattcaacgtatgaaaaacaaaggtttgggagcgtccatcatcaatatgagcagcatccttggattcgtaggggacccgtcgctgggtgcatacaacgcctcgaaaggggcggtgcgcattatgtcaaaaagcgcggccctggactgtgccttaaaagattatgatgtacgcgtgaacacagttcatcccggttacattaaaaccccgcttgtcgatgatctccccggcgaagaggaagcgatgtctcagcgcaccaaaacgccgatgggccacattggcgaacctaacgatatcgcatatatttgcgtttacctggcaagcaatgaatctaaatttgcgaccggctcagagtttgtggtggatggcggctataccgcgcagtaa

SEQ ID NO: 22 - DNA, 759 bp, synthetic construct
  source 1..759: mol_type = other DNA
atgtccaatcgcttggacgggaaggttgcgattattaccggtggcaccctgggcatcggcctggcgatcgctactaaatttgtggaagaaggtgccaaggtcatgattaccggccgtcacagcgatgtaggcgaaaaagcagcaaagtccgtcgggacccctgatcagattcaattctttcaacacgattcgagcgacgaggatggatggactaaattgtttgatgccaccgaaaaggcattcggtcctgtaagtaccttggtcaacaatgcaggcatcgctgtaaacaaaagcgtcgaggagactactacggcagaatggcgcaaacttctggccgtcaacttggacggcgttttttttggcacgcgtctgggcattcaacgtatgaaaaacaaaggtttgggagcgtccatcatcaatatgagcagcatccttggattcgtaggggacccgtcgctgggtgcatacaacgcctcgaaaggggcggtgcgcattatgtcaaaaagcgcggccctggactgtgccttaaaagattatgatgtacgcgtgaacacagttcatcccggttacattaaaaccccgcttgtcgatgatctccccggcattgaggaagcgatgtctcagcgcaccaaaacgccgatgggccacattggcgaacctaacgatatcgcatatatttgcgtttacctggcaagcaatgaatctaaatttgcgaccggctcagagtttgtggtggatggcggctataccgcgcagtaa

SEQ ID NO: 23 - DNA, 759 bp, synthetic construct
  source 1..759: mol_type = other DNA
atgtccaatcgcttggacgggaaggttgcgattattaccggtggcaccctgggcatcggcctggcgatcgctactaaatttgtggaagaaggtgccaaggtcatgattaccggccgtcacagcgatgtaggcgaaaaagcagcaaagtccgtcgggacccctgatcagattcaattctttcaacacgattcgagcgacgaggatggatggactaaattgtttgatgccaccgaaaaggcattcggtcctgtaagtaccttggtcaacaatgcaggcatcgctgtaaacaaaagcgtcgaggagactactacggcagaatggcgcaaacttctggccgtcaacttggacggcgttttttttggcacgcgtctgggcattcaacgtatgaaaaacaaaggtttgggagcgtccatcatcaatatgagcagcatccttggattcgtaggggacccgtcgctgggtgcatacaacgcctcgaaaggggcggtgcgcattatgtcaaaaagcgcggccctggactgtgccttaaaagattatgatgtacgcgtgaacacagttcatcccggttacattaaaaccccgcttgtcgatgatctccccggcaccgaggaagcgatgtctcagcgcaccaaaacgccgatgggccacattggcgaacctaacgatatcgcatatatttgcgtttacctggcaagcaatgaatctaaatttgcgaccggctcagagtttgtggtggatggcggctataccgcgcagtaa

SEQ ID NO: 24 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcattctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatatgccgggcattgaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 25 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcattctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatatgccgggcctggaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 26 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcattctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgataacccgggcctggaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 27 - DNA, 759 bp, synthetic construct
  source 1..759: mol_type = other DNA
atgagcaatcgtctggatggaaaggtagcaattattaccggcgggactctgggcattggactcgcgattgcgacaaaattcgtggaagaaggcgcgaaagtgatgattacgggtcgccattcggacgtaggggaaaaagctgcgaaaagtgttggcactccggaccagattcagttttttcaacatgattcctccgatgaggatggctggacgaaattattcgacgcgaccgaaaaagcatttgggccggtctcaacattggtcaataatgctggcatcgccgtcaataaatctgtcgaagaaaccaccaccgctgaatggcgcaaactgctggccgtcaatctggatggcgttttctttggtacgcggctcgggattcagcggatgaagaacaaagggctgggggcaagtatcattaatatgtcgagcatccttgggtttgtcggcgacccctcattaggggcctacaacgctagcaaaggtgccgtacgcatcatgagcaaatctgcggcgttggactgcgccctgaaagattacgatgtgcgcgttaataccgtccatccgggttatattaaaacgccgttggtagatgataacccaggtatcgaggaagcaatgtcccagcgcaccaaaaccccaatgggacatattggcgaaccgaacgatattgcctatatttgtgtatacctggcgtcaaatgagtctaaatttgcgacggggagcgaatttgtggtagatggcggctacaccgcgcaataa

SEQ ID NO: 28 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcattctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatagcccgggcctggaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 29 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcattgcgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatagcccgggcattgaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 30 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacattagcagcattctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatagcccgggcattgaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 31 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcgcgctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatatgccgggcattgaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 32 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcgcgctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgataacccgggcattgaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 33 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcgcgctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatatgccgggcctggaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

SEQ ID NO: 34 - DNA, 759 bp, synthetic construct
  source 1..759: mol_type = other DNA
atgagcaaccgcctggatggcaaagtggcgattattaccggcggcaccctgggcattggcctggcgattgcgaccaaatttgtggaagaaggcgcgaaagtgatgattaccggccgccatagcgatgtgggcgaaaaagcggcgaaaagcgtgggcaccccggatcagattcagttttttcagcatgatagcagcgatgaagatggctggaccaaactgtttgatgcgaccgaaaaagcgtttggcccggtgagcaccctggtgaacaacgcgggcattgcggtgaacaaaagcgtggaagaaaccaccaccgcggaatggcgcaaactgctggcggtgaacctggatggcgtgttttttggcacccgcctgggcattcagcgcatgaaaaacaaaggcctgggcgcgagcattattaacatgagcagcgcgctgggctttgtgggcgatccaagcttgggcgcgtataacgcgagcaaaggcgcggtgcgcattatgagcaaaagcgcggcgctggattgcgcgctgaaagattatgatgtgcgcgtgaacaccgtgcatccgggctatattaaaaccccgctggtggatgatagcccgggcctggaagaagcgatgagccagcgcaccaaaaccccgatgggccatattggcgaaccgaacgatattgcgtatatttgcgtgtatctggcgagcaacgaaagcaaatttgcgaccggcagcgaatttgtggtggatggcggctataccgcgcagtaa

(19) United States
(12) Patent Application Publication
(10) Pub. No.: US 2025/0154475 A1
(43) Pub. Date: May 15, 2025

(54) MUTANT KETOREDUCTASE WITH INCREASED KETOREDUCTASE ACTIVITY AS WELL AS METHODS AND USES INVOLVING THE SAME

(71) Applicant: Hoffmann-La Roche Inc., Little Falls, NJ (US)

(72) Inventors: Hans Iding, Magden (CH); Irene Marzuoli, Basel (CH)

(21) Appl. No.: 19/022,356

(22) Filed: Jan. 15, 2025

Related U.S. Application Data
(63) Continuation of application No. PCT/EP2023/069686, filed on Jul. 14, 2023.

(30) Foreign Application Priority Data
Jul. 15, 2022 (EP) 22185241.1

(51) Int. Cl. C12N 9/04; C12P 17/14

(52) U.S. Cl. CPC C12N 9/0006; C12P 17/14; C12Y 101/01002

(57) ABSTRACT

The present invention relates to a mutant ketoreductase, a nucleic acid encoding the mutant ketoreductase, a vector comprising the nucleic acid, a method for the enzymatic reduction of a prochiral ketone and the formation of a chiral alcohol with the mutant ketoreductase, the use of the mutant ketoreductase for the preparation of chiral alcohols as well as the use of the method for the preparation of pharmaceutically active morpholine compounds.

The present invention relates to a mutant ketoreductase, a nucleic acid encoding the mutant ketoreductase, a vector comprising the nucleic acid, a method for the enzymatic reduction of a prochiral ketone and the formation of a chiral alcohol with the mutant ketoreductase, the use of the mutant ketoreductase for the preparation of chiral alcohols as well as the use of the method for the preparation of pharmaceutically active morpholine compounds.

Ketoreductases are a subclass of enzymes belonging to the group of oxidoreductases, i.e. enzymes catalyzing redox reactions allowing the transfer of an electron from a so-called electron donor molecule to an electron acceptor molecule.

The subclass of ketoreductases have the specific capability of catalyzing the enantioselective conversion of desired prochiral ketones to their corresponding secondary alcohol. During this reduction reaction, electrons are transferred to the ketone group (C═O) leading to the addition of a first hydrogen to the carbon of the ketogroup and a second hydrogen to the oxygen. This reaction generally requires electron donors as cofactors, such as NADH or NADPH, which may be regenerated in-situ.

By now, ketoreductases are also commonly used in the enzymatic reduction of prochiral keto compounds and accordingly for the preparation of intermediates for various pharmaceutical compounds, such as for the preparation of compounds that have a good affinity to Trace amine-associated receptors (TAARs).

TAARs belong to the group of G protein-coupled receptors and function as receptors for various endogenous and exogenous compounds. There are six functional human TAARs known. One of them is TAARI, which has been identified as being a receptor for metabolic derivatives, such as those of the amino acids phenylalanine, tyrosine and tryptophan. Moreover, TAARI works as a receptor for exogenous compounds, such as ephedrine or synthetic psychostimulants, e.g. amphetamine and metamphetamine.

The chiral alcohols of formula I

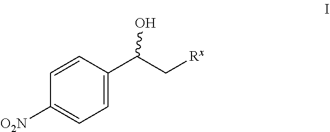

wherein R^{X} stands for hydrogen, C₁₋₄ alkyl or for a halogen atom, are key intermediates for the preparation of compounds that have a good affinity to the TAARs, especially for TAARI as for instance outlined in PCT Publications WO 2012/016879, WO 2012//126922 and WO 2017/157873. Such compounds may for example be used in the treatment of psychiatric disorders, such as schizophrenia and mood disorders.

A particular promising TAARI clinical candidate is ralmitaront, which has the formula X.

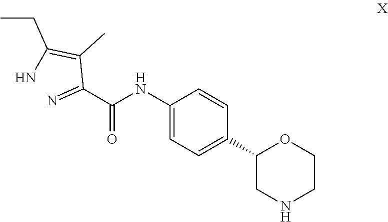 [SMILES: CC/C(N)=C(\C)C(=N)C(=O)Nc1ccc([C@H]2CNCCO2)cc1]

The preparation of chiral alcohols of formula I is e.g. described in WO 2015/086495.

It is an object of the present invention to design improved mutant ketoreductases with an increased ketoreductase activity relative to the wildtype ketoreductase, particularly the ketoreductase of Lactobacillus brevis, especially that of SEQ ID NO: 1. These mutants may be used in the production of chiral alcohols, such as chiral alcohols of formula I, including its production in an scaled-up process.

Surprisingly, it has been found that a mutant ketoreductase comprising an amino acid sequence that is at least 80% identical to the amino acid sequence of SEQ ID NO: 1 (Lactobacillus brevis ATCC 14869 ketoreductase, referred to as Q84EX5 in UniProtKB); and wherein the mutant ketoreductase has at least two amino acid substitutions relative to the amino acid sequence of SEQ ID NO: 1, wherein the amino acid at the position corresponding to position 145 of SEQ ID NO: 1 is substituted with Leu, Ala, Cys, Met or Thr (Leu145, Ala145, Cys145, Met145 or Thr145, respectively) and the amino acid at the position corresponding to position 202 of SEQ ID NO: 1 is substituted with Cys, Glu, Ile, Leu or Thr (Cys202, Glu202, Ile202, Leu202 15 or Thr202, respectively) shows an increased ketoreductase activity relative to the wildtype ketoreductase, particularly the ketoreductase of Lactobacillus brevis, especially that of SEQ ID NO: 1.

As shown in the examples, various single and double mutations introduced into the ketoreductase of Lactobacillus brevis, especially that of SEQ ID NO: 1, increased the ketoreductase activity relative to the wildtype, as may be taken from the values of Fold Improvement Over the Parent (FIOP) (see Tables 1 and 2). The combination of mutations at sites 145 and 202 has been proven as particularly suitable. At position 145 substitutions with Leu, Ala, Cys, Met or Thr (Leu145, Ala145, Cys145, Met145 or Thr145, respectively) increased ketoreductase activity significantly (>8 fold as shown in Table 2). An additional mutation at position 202, i.e. substitution with Cys, Glu, Ile, Leu or Thr (Cys202, Glu202, Ile202, Leu202 or Thr202, respectively), further increased the activity (>11 fold as shown in Table 2). Further mutations may be added such as substitutions at positions 141, 144 or 199 (see Tables 3 to 7). Moreover, mutations at position 16 and 43 showed beneficial effects as well (see Tables 1 and 2).

In addition, it has been found that such ketoreductase are highly active for catalysing the enzymatic reduction of prochiral ketones and for the formation of chiral alcohols which may serve as intermediates in the preparation of compounds that have a good affinity to the TAARs, such as TAAR1.

Moreover, it has been found that the mutant ketoreductase of the present invention in comparison to the wild-type ketoreductase catalyzes the enzymatic reduction of prochiral ketones with an increased selectivity and an increased conversion.

Especially it has been found that the enzymatic reduction of a ketone of the formula

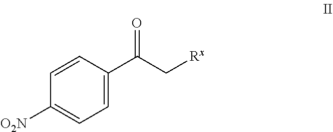

wherein R^{x} is hydrogen, C₁₋₄ alkyl or a halogen atom with the mutant ketoreductase forms the chiral alcohol of the formula

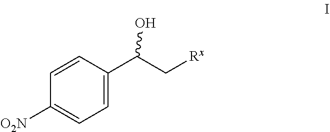

wherein R^{x} is as above, with an extraordinary high enantiomeric excess. Therefore, the ketoreductase according to the present invention is extremely useful for the preparation of key intermediates, particularly for key intermediates in the preparation of compounds that have a good affinity to the TAARs, such as TAARI and more particularly for TAARI clinical candidate of the formula X

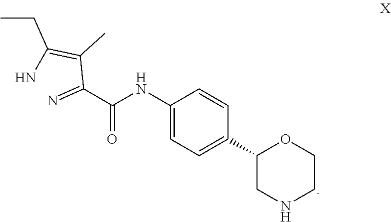 [SMILES: CC/C(N)=C(\C)C(=N)C(=O)Nc1ccc([C@H]2CNCCO2)cc1]

Accordingly, in a first aspect the present invention relates to a mutant ketoreductase with increased ketoreductase activity relative to the wild-type ketoreductase, wherein the mutant ketoreductase comprises an amino acid sequence that is at least 80% identical to the amino acid sequence of SEQ ID NO: 1 (Lactobacillus brevis ATCC 14869 ketoreductase); and
wherein the mutant ketoreductase has at least two amino acid substitutions relative to the amino acid sequence of SEQ ID NO: 1, wherein
   the amino acid at the position corresponding to position 145 of SEQ ID NO: 1 is substituted with Leu, Ala, Cys, Met or Thr (Leu145, Ala145, Cys145, Met145 or Thr145, respectively) and
   the amino acid at the position corresponding to position 202 of SEQ ID NO: 1 is substituted with Cys, Glu, Ile, Leu or Thr (Cys202, Glu202, Ile202, Leu202 or Thr202, respectively).

The term “ketoreductase” as used herein means any protein having the capability of catalyzing the enantioselective conversion of a prochiral ketone to the corresponding secondary alcohol.

The term “wild-type ketoreductase” as used herein means any ketoreductase which occurs as such in nature. The term “mutant ketoreductase” as used herein means any ketoreductase, which originates from a corresponding wild-type ketoreductase and in comparison to such wild-type ketoreductase has been amended in its amino acid sequence. For example, this may comprise the introduction, deletion, substitution or post-translational mutation of one or more amino acids at one or more positions. Preferably, the mutant ketoreductase differs from the wild-type ketoreductase by amino acid substitutions. Methods for creating mutations, such as amino acid substitutions, in amino acid sequences are well-known to the person skilled in the art. For example, such mutations may already be introduced on nucleic acid level leading to the expression of the desired mutated amino acid sequence. Suitable methods therefore are well-known to the person skilled in the art and partly also described below, e.g. in the context of nucleic acids according to the second aspect of the invention.

Suitable mutant ketoreductases according to the first aspect may originate from the wild-type ketoreductase of any organism. A preferred source is Lactobacillus brevis. Especially preferred is the Lactobacillus brevis ATCC 14869 ketoreductase, referred to as Q84EX5.

In accordance with the present invention, the mutant ketoreductase is active as ketoreductase. This means that the mutant ketoreductase is capable of converting a prochiral ketone to the corresponding secondary alcohol under suitable conditions, as detailed above and below. Methods for determining ketoreductase activity are described herein and given in the Examples.

The mutant ketoreductase according to the first aspect shows increased ketoreductase activity relative to the wild-type ketoreductase.

The activity may be determined in an enzyme assay measuring either the consumption of substrate or production of product over time. A large number of different methods of measuring the concentrations of substrates and products exist and many enzymes can be assayed in several different ways as known to the person skilled in the art.

Methods of determining enzymatic activity of a mutant ketoreductase according to the present invention or a wild-type ketoreductase are well-known to the person skilled in the art. Exemplary methods are also described in the Examples. To determine, whether a mutant ketoreductase according to the first aspect shows increased ketoreductase activity relative to the wild-type ketoreductase, ketoreductase activity of both ketoreductases is measured using the same method.

For example, methods of determining enzymatic activity of a ketoreductase in general may be based on a fluorescence or colorimetric assay. Further, methods of determining enzymatic activity of a ketoreductase in general may comprise the detection of the concentration of product being formed, the educt consumed or the detection of formed or consumed cofactors necessary for the reaction, such as the concentrations of NAD⁺, NADH, NADP⁺ or NADPH.

A mutant ketoreductase according to the first aspect showing increased ketoreductase activity relative to the wild-type ketoreductase, for example, shows an increase in ketoreductase activity by more than the onefold. The person skilled in the art knows statistical procedures to assess whether or not one value of enzyme activity is increased relative to another, such as Student's t-test or chi-square test. It is evident for the skilled person that any background signal has to be subtracted when analyzing the data.

In addition to an increased ketoreductase activity, the mutant ketoreductase according to the first aspect may further relative to the wild-type ketoreductase have an increased selectivity. The term “The term “selectivity” as used herein, means the portion of the total reacted substrate that has been converted into the desired target product, taking into account stoichiometry. In general, the selectivity of an enzyme, such as a ketoreductase, may be affected by various reaction parameters, such as temperature, pressure, concentrations, solvents or reaction time. Methods for determining the selectivity of the mutant ketoreductase as well as the wild-type ketoreductase are well-known to the person skilled in the art, such as by analyzing the substrate remaining after enzyme reaction.

Further, the mutant ketoreductase according to the first aspect of the invention comprises an amino acid sequence that is at least 80% identical to the amino acid sequence of SEQ ID NO: 1 (Lactobacillus brevis ATCC 14869 ketoreductase, referred to as Q84EX5 in UniProtKB). Thereby, the amino acid sequence of SEQ ID NO: 1 originates from Lactobacillus brevis strain ATCC 14869 ketoreductase, which is referred to as Q84EX5 in UniProtKB.

The term “sequence identity” as used herein describes the percentage of characters that exactly match between two different sequences.

For example, the term “at least 80% identical to the amino acid sequence of SEQ ID NO: 1” as used herein means that the amino acid sequence of the mutant ketoreductase of the present invention has an amino acid sequence characterized in that, within a stretch of 100 amino acids, at least 80 amino acid residues are identical to the sequence of the corresponding sequence of SEQ ID NO: 1.

The mutant ketoreductase may also comprise an amino acid sequence that is at least 85%, 90%, 95%, 96%, 97%, 98% or 99% identical to the amino acid sequence of SEQ ID NO: 1 (Lactobacillus brevis ATCC 14869 ketoreductase, referred to as Q84EX5 in UniProtKB or L. brevis-Rad).

Sequence identity according to the present invention can, e.g., be determined by methods of sequence alignment in form of sequence comparison. Methods of sequence alignment are well known in the art and include various programs and alignment algorithms. Moreover, the NCBI Basic Local Alignment Search Tool (BLAST) is available from several sources, including the National Center for Biotechnology Information (NCBI, Bethesda, MD) and on the internet, for use in connection with the sequence analysis programs blastp, blastn, blastx, tblastn and tblastx. Percentage of identity of mutants according to the present invention relative to the amino acid sequence of e.g. SEQ ID NO: 1 is typically characterized using the NCBI Blast blastp with standard settings. Alternatively, sequence identity may be determined using the software GENEious with standard settings. Alignment results can be, e.g., derived from the Software Geneious (version R8), using the global alignment protocol with free end gaps as alignment type, and Blosum62 as a cost matrix.

The mutant ketoreductase according to the present invention has at least two amino acid substitutions relative to the amino acid sequence of SEQ ID NO: 1. Moreover, the mutant ketoreductase according to the present invention may have at least three, four, five, six, seven, eight, nine, ten or more amino acid substitutions relative to the amino acid sequence of SEQ ID NO: 1.

Especially, the mutant ketoreductase according to the present invention has at least two amino acid substitutions relative to the amino acid sequence of SEQ ID NO: 1, wherein
the amino acid at the position corresponding to position 145 of SEQ ID NO: 1 is substituted with Leu, Ala, Cys, Met or Thr (Leu145, Ala145, Cys145, Met145 or Thr145, respectively) and
the amino acid at the position corresponding to position 202 of SEQ ID NO: 1 is substituted with Cys, Glu, Ile, Leu or Thr (Cys202, Glu202, Ile202, Leu202 or Thr202, respectively).

Methods for preparing the mutant ketoreductase according to the first aspect of the present invention are well-known to the person skilled in the art. For example, the mutant ketoreductase according to the first aspect may be prepared by using any method suitable for preparing a recombinant enzyme known to the person skilled in the art, such as recombinant expression of the modified nucleic acid of the mutant ketoreductase in cell culture, followed by protein isolation and purification.

Preferably, in the mutant ketoreductase of the first aspect, the amino acid at the position corresponding to position 145 of SEQ ID NO: 1 is substituted with Leu (Leu145). Also preferably, in the mutant ketoreductase of the first aspect the amino acid at the position corresponding to position 202 of SEQ ID NO: 1 is substituted with Ile (Ile202).

Even more preferred, in the mutant ketoreductase of the first aspect, the amino acid at the position corresponding to position 145 of SEQ ID NO: 1 is substituted with Leu (Leu145) and the amino acid at the position corresponding to position 202 of SEQ ID NO: 1 is substituted with Ile (Ile202).

In a preferred embodiment of the mutant ketoreductase of the first aspect,
the amino acid at the position corresponding to position 145 of SEQ ID NO: 1 is substituted with Leu (Leu145) and/or
the amino acid at the position corresponding to position 202 of SEQ ID NO: 1 is substituted with Ile (Ile202) or Leu (Leu202), preferably wherein the amino acid at the position corresponding to position 145 of SEQ ID NO: 1 is substituted with Leu (Leu145) and the amino acid at the position corresponding to position 202 of SEQ ID NO: 1 is substituted with Ile (Ile202) or wherein the amino acid at the position corresponding to position 145 of SEQ ID NO: 1 is substituted with Leu (Leu145) and the amino acid at the position corresponding to position 202 of SEQ ID NO: 1 is substituted with Leu (Leu202).

If the mutant ketoreductase according to the first aspect has more than two amino acid substitutions relative to the amino acid sequence of SEQ ID NO: 1, such as three, four, five, six, seven, eight, nine, ten or more amino acid substitutions relative to the amino acid sequence of SEQ ID NO: 1, the amino acid sequence of the mutant ketoreductase of the first aspect of the invention preferably comprises substitutions at positions corresponding to positions 16, 43, 141, 144 and/or 199, of SEQ ID NO: 1 in addition to those at positions 145 and 202.

Especially preferred are mutations Leu145 and Ile202 in combination with
Asn199;
Met199;
Ser199;
Ile141 and Ser199;
Ile141 and Asn199;
Ala144 and Asn199; or
Ala144 and Met199, or
   mutations Leu145 and Leu202 in combination with
Asn199;
Met199;
Ser199;
Ile141 and Ser199;
Ile141 and Asn199;
Arg144 and Met199; or
Arg144 and Ser199.

Further mutations, optionally as defined above or below, may be present.

If the mutant ketoreductase according to the first aspect has an amino acid substitution at the position corresponding to position 16 of SEQ ID NO: 1, the position corresponding to position 16 of SEQ ID NO: 1 is preferably substituted with Ala, Cys, Gly, Ile, Met, Ser, Tyr or Val (Ala16, Cys16, Gly16, Ile16, Met16, Ser16, Tyr16 or Val16, respectively). More preferably, in such case, in the mutant ketoreductase of the first aspect, the amino acid at the position corresponding to position 16 is substituted with Ala, Gly, Ile, Ser or Tyr (Ala16, Gly16, Ile16, Ser16, or Tyr16, respectively). Most preferably, in such case, in the mutant ketoreductase of the first aspect, the amino acid at the position corresponding to position 16 is substituted with Ala, Gly or Tyr (Ala16, Gly16, Tyr16, respectively).

If the mutant ketoreductase according to the first aspect has an amino acid substitution at the position corresponding to position 43 of SEQ ID NO: 1, the amino acid at the position corresponding to position 43 is preferably substituted with Ile (Gln43) or Lys (Lys43).

If the mutant ketoreductase according to the first aspect has an amino acid substitution at the position corresponding to position 141 of SEQ ID NO: 1, the amino acid at the position corresponding to position 141 is preferably substituted with Ile (Ile141).

If the mutant ketoreductase according to the first aspect has an amino acid substitution at the position corresponding to position 144 of SEQ ID NO: 1, the amino acid at the position corresponding to position 144 is preferably substituted with Ala, Cys, Ser, Thr or Val (Ala144, Cys144, Ser144, Thr144 or Val144, respectively). More preferably, in such case, in the mutant ketoreductase of the first aspect the amino acid at the position corresponding to position 144 is substituted with Ala (Ala144).

If the mutant ketoreductase according to the first aspect has an amino acid substitution at the position corresponding to position 199 of SEQ ID NO: 1, the amino acid at the position corresponding to position 199 is substituted with Phe, Met, Gin, Ser or Val (Phe199, Met199, Gln199, Ser199 or Val199, respectively). More preferably, in such case, in the mutant ketoreductase of the first aspect, the amino acid at the position corresponding to position 199 is substituted with Gin, Met or Ser (Gln199, Met199 or Ser199, respectively).

In a further preferred embodiment of the mutant ketoreductase of the first aspect,
the amino acid at the position corresponding to position 16 is substituted with Ala, Cys, Gly, Ile, Met, Ser, Tyr or Val (Ala16, Cys16, Gly16, Ile16, Met16, Ser16, Tyr 16 or Val16, respectively), preferably with Ala, Gly, Ile, Ser or Tyr (Ala16, Gly16, Ile16, Ser16 or Tyr16, respectively), more preferably with Ala, Gly or Ser (Ala16, Gly16, Ser16, respectively); and/or
the amino acid at the position corresponding to position 43 is substituted with Gln or Lys (Gln43 or Lys43); and/or
the amino acid at the position corresponding to position 141 is substituted with Ile (Ile141); and/or
the amino acid at the position corresponding to position 144 is substituted with Ala, Cys, Ser, Thr or Val (Ala144, Cys144, Ser144, Thr144 or Val144, respectively), preferably wherein the amino acid at the position corresponding to position 144 is substituted with Ala (Ala144); and/or
the amino acid at the position corresponding to position 199 is substituted with Asn, Phe, Met, Gln, Ser or Val (Phe199, Met199, Gln199, Ser199 or Val199, respectively), preferably Asn, Gln, Met or Ser (Asn199, Gln199, Met199 or Ser199, respectively).

Increased activity has been proven for mutant ketoreductases having the following substitutions:
Leu145 in combination with Cys202, Glu202, Ile202 or Thr202; or
Leu145 and Ile202 in combination with
   Asn199, Met199, or Ser199; or
Leu145 and Leu202 in combination with
   Asn199, Met199, or Ser199; or
Leu145 and Ile202 in combination with
   Ala144 and Asn199,
   Ala144 and Met199,
   Ile141 and Ser199; or
Leu145 and Leu202 in combination with
   Ala144 and Met199,
   Ala144 and Ser199.

If the mutant ketoreductase according to the first aspect has more than two amino acid substitutions relative to the amino acid sequence of SEQ ID NO: 1, such as three, four, five, six, seven, eight, nine, ten or more amino acid substitutions relative to the amino acid sequence of SEQ ID NO: 1, the amino acid sequence of the mutant ketoreductase of the first aspect of the invention may comprise substitutions at positions corresponding to position 145, 199 and 202 of SEQ ID NO: 1, optionally in combination with position 141.

In a preferred embodiment of the mutant ketoreductase of the first aspect,
the amino acid at the position corresponding to position 145 of SEQ ID NO: 1 is substituted with Leu (Leu145) and
the amino acid at the position corresponding to position 199 of SEQ ID NO: 1 is substituted with Asn (Asn199) and
the amino acid at the position corresponding to position 202 of SEQ ID NO: 1 is substituted with Ile (Ile202).

In an also preferred embodiment of the mutant ketoreductase of the first aspect,
the amino acid at the position corresponding to position 145 of SEQ ID NO: 1 is substituted with Leu (Leu145) and
the amino acid at the position corresponding to position 199 of SEQ ID NO: 1 is substituted with Ser (Ser199) and
the amino acid at the position corresponding to position 202 of SEQ ID NO: 1 is substituted with Ile (Ile202).

In an even more preferred embodiment of the mutant ketoreductase of the first aspect,
the amino acid at the position corresponding to position 141 of SEQ ID NO: 1 is substituted with Ile (Ile141) and
the amino acid at the position corresponding to position 145 of SEQ ID NO: 1 is substituted with Leu (Leu145) and
the amino acid at the position corresponding to position 199 of SEQ ID NO: 1 is substituted with Asn (Asn199) and
the amino acid at the position corresponding to position 202 of SEQ ID NO: 1 is substituted with Ile (Ile202).

Most preferably, the mutant ketoreductase is characterized in that
the amino acid at the position corresponding to position 141 of SEQ ID NO: 1 is substituted with Ile (Ile141) and
the amino acid at the position corresponding to position 145 of SEQ ID NO: 1 is substituted with Leu (Leu145) and
the amino acid at the position corresponding to position 199 of SEQ ID NO: 1 is substituted with Ser (Ser199) and
the amino acid at the position corresponding to position 202 of SEQ ID NO: 1 is substituted with Ile (Ile202).

In an additional preferred embodiment of the mutant ketoreductase of the first aspect, the mutant ketoreductase does not comprise a mutation at one or more of portions 94, 96, 153, 190, 195, 206 and 233. Thereby, the expression “does not comprise a mutation at one or more of positions corresponding to position 94, 96, 153, 190, 195, 206 and 233 of SEQ ID NO: 1” as used herein means that the amino acid of the mutant ketoreductase according to the first aspect at one or more of positions corresponding to SEQ ID NO: 1 94, 96, 153, 190, 195, 206 and 233 corresponds to that of SEQ ID NO: 1.

For example, the mutant ketoreductase according to the first aspect does not comprise a mutation at one, two, three, four, five, six or seven positions corresponding to positions 94, 96, 153, 190, 195, 206 and 233 of SEQ ID NO: 1.

The mutant ketoreductase according to the first aspect of the invention may further comprise an amino acid sequence that is at least 80%, 85%, 90%, 91%, 92%, 93%, 94%, 95%, 96%, 97%, 98%, or 99%, particularly 100% identical to the amino acid sequence of SEQ ID NO: 2 to 17. Sequence identity and methods for determining sequence identity of the amino acid sequences of two proteins are well-known to the person skilled in the art and also described above.

In a further preferred embodiment of the mutant ketoreductase of the first aspect, the mutant ketoreductase consists of or comprises an amino acid sequence that is at least 85%, 90%, 91%, 92%, 93%, 94%, 95%, 96%, 97%, 98%, or 99%, particularly 100% identical to the amino acid sequence of any of SEQ ID NO: 2 to 17.

In a preferred embodiment of the mutant ketoreductase of the first aspect, the ketoreductase activity relative to the wild-type ketoreductase is increased by at least 2.0, 5.0, or 10-fold. Methods for determining the ketoreductase activity of a protein as well as methods for comparing the ketoreductase activity of two or more proteins are well-known to the person skilled in the art and also described above.

The mutant ketoreductase according to the first aspect of the invention may further have an increased conversion relative to the wild-type ketoreductase at 10% substrate loading and at a mutant or wild-type ketoreductase loading of 1% [w/w] (s/e 100) using 2-Propanol recycling system.

The term “conversion” as used herein means any substrate to product conversion induced by a ketoreductase, such as by a mutant ketoreductase of the present invention or a wild-type ketoreductase. Such conversion may further depend on various reaction parameters, such as temperature, pressure or the amount of used substrate or ketoreductase enzyme. Suitable conditions and methods are described in the Examples (see Examples 2 to 4).

Preferably, the conversion of a ketoreductase is determined at 10% substrate loading and at a mutant or wild-type ketoreductase loading of 1% [w/w] (s/e 100) using 2-Propanol recycling system. In this context, the abbreviation “s/e” describes the term “substrate/enzyme”. A loading of 1% [w/w] (s/e 100) further means that 100 g substrate are used per 1 g enzyme, i.e. substrate and enzyme are used in a ratio of 100/1.

In a further preferred embodiment of the mutant ketoreductase of the first aspect, the mutant ketoreductase has an increased conversion relative to the wild-type ketoreductase at 10% substrate loading and at a mutant or wild-type ketoreductase loading of 1% [w/w] (s/e 100) using 2-Propanol recycling system, particularly an increased conversion of at least 2.0, 5.0, 7.5, or 10-fold.

In a preferred embodiment of the mutant ketoreductase of the first aspect, the mutant ketoreductase is capable of converting a prochiral ketone into a chiral alcohol.

In a further preferred embodiment the prochiral ketone has the formula II

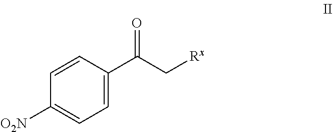

wherein R^{x} is hydrogen, C₁₋₄ alkyl or a halogen atom and the resulting chiral alcohol has the formula I

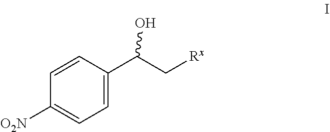

wherein Rx is hydrogen, C₁₋₄ alkyl or a halogen atom.

The spiral bond
“ [symbol] ”

stands for “ [symbol] ” or for “ [symbol] ” thus indicating chirality of the molecule.

Suitable C₁₋₄ alkyl groups are selected from methyl, ethyl, n-propyl, i-propyl, n-Butyl, i-butyl or t-butyl, preferably from methyl.

Suitable halogen atoms are fluorine, chlorine, bromine and iodine, but preferably is bromine.

The mutant ketoreductase can in principle catalyze the formation of both the S- and the R-enantiomer of a chiral alcohol, particularly of the chiral alcohol of formula I.

In a preferred embodiment the mutant ketoreductase catalyzes for the formation of the S-enantiomer of the chiral alcohol of formula Ia

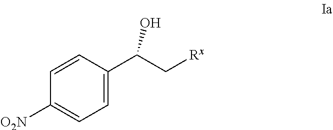

wherein R^{x} is hydrogen, C₁₋₄ alkyl or a halogen atom, more preferably for the formation of the chiral alcohol of formula Ib

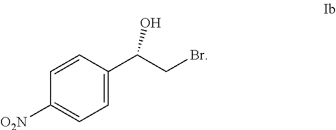 [SMILES: O=[N+]([O-])c1ccc([C@H](O)CBr)cc1]

An enantiomeric excess of the S-enantiomer of the chiral alcohol of at least 95%, 96%, 97%, 98% or 99% can be reached.

Further, the mutant ketoreductase according to the first aspect of the invention may also be combined with a further peptide or protein into a fusion protein. Accordingly, the present invention further concerns a fusion protein comprising the mutant ketoreductase of the present invention.

The fusion protein may further comprise a tag. Tags are attached to proteins for various purposes, e.g. in order to ease purification, to assist in the proper folding in proteins, to prevent precipitation of the protein, to alter chromatographic properties, to modify the protein or to mark or label the protein. A number of (affinity) tags or (affinity) markers are known at present. Commonly used tags include the Arg-tag, the His-tag, the Strep-tag, the Flag-tag, the T7-tag, the S-tag, the HAT-tag, the GST-tag and the MBP-tag.

In a second aspect the present invention relates to a nucleic acid coding for the mutant ketoreductase according to the first aspect of the invention. Accordingly, the present invention may also relate to a nucleic acid coding for the fusion protein comprising the mutant ketoreductase according to the first aspect of the invention.

The term “nucleic acid” as used herein generally relates to any nucleotide molecule which encodes the mutant ketoreductase of the invention and which may be of variable length. Examples of a nucleic acid of the invention include, but are not limited to, plasmids, vectors, or any kind of DNA and/or RNA fragment(s) which can be isolated by standard molecular biology procedures, including, e.g. ion-exchange chromatography. A nucleic acid of the invention may be used for transfection or transduction of a particular cell or organism.

Nucleic acid molecule of the present invention may be in the form of RNA, such as mRNA or cRNA, or in the form of DNA, including, for instance, cDNA and genomic DNA e.g. obtained by cloning or produced by chemical synthetic techniques or by a combination thereof. The DNA may be triple-stranded, double-stranded or single-stranded. Single-stranded DNA may be the coding strand, also known as the sense strand, or it may be the non-coding strand, also referred to as the anti-sense strand. Nucleic acid molecule as used herein also refers to, among other, single- and double-stranded DNA, DNA that is a mixture of single- and double-stranded RNA, and RNA that is a mixture of single- and double-stranded regions, hybrid molecules comprising DNA and RNA that may be single-stranded or, more typically, double-stranded, or triple-stranded, or a mixture of single- and double-stranded regions. In addition, nucleic acid molecule as used herein refers to triple-stranded regions comprising RNA or DNA or both RNA and DNA.

Additionally, the nucleic acid may contain one or more modified bases. Such nucleic acids may also contain modifications e.g. in the ribose-phosphate backbone to increase stability and half life of such molecules in physiological environments. Thus, DNAs or RNAs with backbones modified for stability or for other reasons are “nucleic acid molecule” as that feature is intended herein. Moreover, DNAs or RNAs comprising unusual bases, such as inosine, or modified bases, such as tritylated bases, to name just two examples, are nucleic acid molecule within the context of the present invention. It will be appreciated that a great variety of modifications have been made to DNA and RNA that serve many useful purposes known to those of skill in the art. The term nucleic acid molecule as it is employed herein embraces such chemically, enzymatically or metabolically modified forms of nucleic acid molecule, as well as the chemical forms of DNA and RNA characteristic of viruses and cells, including simple and complex cells, inter alia.

Furthermore, the nucleic acid molecule encoding the mutant ketoreductase of the invention can be functionally linked, using standard techniques such as standard cloning techniques, to any desired sequence, such as a regulatory sequence, leader sequence, heterologous marker sequence or a heterologous coding sequence to create a fusion protein.

The nucleic acid of the invention may be originally formed in vitro or in a cell in culture, in general, by the manipulation of nucleic acids by endonucleases and/or exonucleases and/or polymerases and/or ligases and/or recombinases or other methods known to the skilled practitioner to produce the nucleic acids.

The nucleic acid of the invention may be comprised in an expression vector, wherein the nucleic acid is operably linked to a promoter sequence capable of promoting the expression of the nucleic acid in a host cell.

In a preferred embodiment of the nucleic acid codes for a mutant ketoreductase of the first aspect, wherein the mutant ketoreductase consists of or comprises an amino acid sequence that is at least 85%, 90%, 91%, 92%, 93%, 94%, 95%, 96%, 97%, 98%, or 99%, particularly 100% identical to the amino acid sequence of any of SEQ ID NO: 2 to 17. Preferably, the nucleic acid has or comprises the sequence of any of SEQ ID NO: 18 to 34.

In a third aspect the present invention relates to a vector comprising a nucleic acid according to the second aspect of the invention. Accordingly, the present invention may also relate to a vector comprising a nucleic acid coding for the fusion protein comprising the mutant ketoreductase according to the first aspect of the invention.

As used herein, the term “vector” generally refers to any kind of nucleic acid molecule that can be used to express a protein of interest in a cell (see also above details on the nucleic acids of the present invention). In particular, the vector of the invention can be any plasmid or vector known to the person skilled in the art which is suitable for expressing a protein in a particular host cell including, but not limited to, mammalian cells, bacterial cell, and yeast cells. A vector of the present invention may also be a nucleic acid which encodes a mutant ketoreductase of the invention, and which is used for subsequent cloning into a respective vector to ensure expression. Plasmids and vectors for protein expression are well known in the art, and can be commercially purchased from diverse suppliers including, e.g., Promega (Madison, WI, USA), Qiagen (Hilden, Germany), Invitrogen (Carlsbad, CA, USA), or MoBiTec (Germany). Methods of protein expression are well known to the person skilled in the art and are, e.g., described in Sambrook et al., 2000, Molecular Cloning: A laboratory manual, Third Edition.

The vector may additionally include nucleic acid sequences that permit it to replicate in the host cell, such as an origin of replication, one or more therapeutic genes and/or selectable marker genes and other genetic elements known in the art such as regulatory elements directing transcription, translation and/or secretion of the encoded protein. The vector may be used to transduce, transform or infect a cell, thereby causing the cell to express nucleic acids and/or proteins other than those native to the cell. The vector optionally includes materials to aid in achieving entry of the nucleic acid into the cell, such as a viral particle, liposome, protein coating or the like. Numerous types of appropriate expression vectors are known in the art for protein expression, by standard molecular biology techniques. Such vectors are selected from among conventional vector types including insects, e.g., baculovirus expression, or yeast, fungal, bacterial or viral expression systems. Other appropriate vectors, of which numerous types are known in the art, can also be used for this purpose. Methods for obtaining such vectors are well-known (see, e.g. Sambrook et al, supra).

As detailed above, the nucleic acid which encodes a mutant ketoreductase of the invention is operably linked to sequence which is suitable for driving the expression of a protein in a host cell, in order to ensure expression of the protein. However, it is encompassed within the present invention that the claimed vector may represent an intermediate product, which is subsequently cloned into a suitable vector to ensure expression of the protein. The vector of the present invention may further comprise all kind of nucleic acid sequences, including, but not limited to, polyadenylation signals, splice donor and splice acceptor signals, intervening sequences, transcriptional enhancer sequences, translational enhancer sequences, drug resistance gene(s) or alike. Optionally, the drug resistance gene may be operably linked to an internal ribosome entry site (IRES), which might be either cell cycle-specific or cell cycle-independent.

The term “operably linked” as used herein generally means that the gene elements are arranged as such that they function in concert for their intended purposes, e.g. in that transcription is initiated by the promoter and proceeds through the DNA sequence encoding the mutant ketoreductase of the present invention. That is, RNA polymerase transcribes the sequence encoding the mutant ketoreductase into mRNA, which in then spliced and translated into a protein.

The term “promoter sequence” as used in the context of the present invention generally refers to any kind of regulatory DNA sequence operably linked to a downstream coding sequence, wherein said promoter is capable of binding RNA polymerase and initiating transcription of the encoded open reading frame in a cell, thereby driving the expression of said downstream coding sequence. The promoter sequence of the present invention can be any kind of promoter sequence known to the person skilled in the art, including, but not limited to, constitutive promoters, inducible promoters, cell cycle-specific promoters, and cell type-specific promoters.

Moreover, the present invention also comprises a host cell comprising the mutant ketoreductase of the present invention or a fusion protein thereof, the nucleic acid of the second aspect of the invention or the vector of the third aspect of the invention.

A “host cell” of the present invention can be any kind of organism suitable for application in recombinant DNA technology, and includes, but is not limited to, all sorts of bacterial and yeast strain which are suitable for expressing one or more recombinant protein(s). Examples of host cells include, for example, various Bacillus subtilis or E. coli strains. A variety of E. coli bacterial host cells are known to a person skilled in the art and include, but are not limited to, strains such as DH5-alpha, HB101, MV1190, JM109, JM101, or XL-1 blue which can be commercially purchased from diverse suppliers including, e.g., Stratagene (CA, USA), Promega (WI, USA) or Qiagen (Hilden, Germany). A particularly suitable host cell is also described in the Examples, namely E. coli BL21 (DE3) cells. Bacillus subtilis strains which can be used as a host cell include, e.g., 1012 wild type: leuA8 metB5 trpC2 hsdRM1 and 168 Marburg: trpC2 (Trp−), which are, e.g., commercially available from MoBiTec (Germany).

The cultivation of host cells according to the invention is a routine procedure known to the person skilled in the art. That is, a nucleic acid encoding a mutant ketoreductase of the invention can be introduced into a suitable host cell(s) to produce the respective protein by recombinant means. These host cells can by any kind of suitable cells, preferably bacterial cells such as E. coli, which can be cultivated in culture. At a first step, this approach may include the cloning of the respective gene into a suitable vector, such as a vector according to the second aspect of the present invention. Vectors are widely used for gene cloning, and can be easily introduced, i.e. transfected, into bacterial cells which have been made transiently permeable to DNA. After the protein has been expressed in the respective host cell, the cells can be harvested and serve as the starting material for the preparation of a cell extract containing the protein of interest. A cell extract containing the protein of interest is obtained by lysis of the cells. Methods of preparing a cell extract by means of either chemical or mechanical cell lysis are well known to the person skilled in the art, and include, but are not limited to, e.g. hypotonic salt treatment, homogenization, or ultrasonification.

In a fourth aspect the present invention relates to a method for the enzymatic reduction of a prochiral ketone and the formation of a chiral alcohol in the presence of mutant ketoreductase of the present invention.

In a preferred aspect the prochiral ketone has the formula II

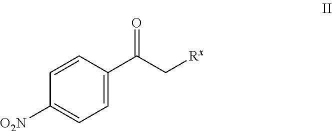

wherein R^{x} is hydrogen, C₁₋₄ alkyl or a halogen atom and the resulting chiral alcohol has the formula I

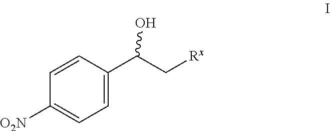

wherein R^{x} is hydrogen, C₁₋₄ alkyl or a halogen atom.

In a further preferred embodiment the S-enantiomer of the chiral alcohol of formula Ia,

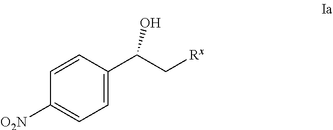

wherein R^{x} is hydrogen, C₁₋₄ alkyl or a halogen atom, more preferably the S-enantiomer of the chiral alcohol of formula Ib,

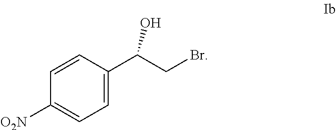 [SMILES: O=[N+]([O-])c1ccc([C@H](O)CBr)cc1]

is prepared.

The 2-bromo-1-(4-nitro-phenyl) ethanone is the ketone of formula II particularly used.

The enzymatic reduction with the mutant ketoreductase usually take place in the presence of NADP as cofactor, which is regenerated in-situ.

The oxidized cofactor is as a rule continuously regenerated with a secondary alcohol as cosubstrate. Typical cosubstrates can be selected from 2-propanol, 2-butanol, pentan-1,4-diol, 2-pentanol, 4-methyl-2-pentanol, 2-heptanol, hexan-1,5-diol, 2-heptanol or 2-octanol, preferably 2-propanol.

Preferably, the cofactor is regenerated by means of the cosubstrate at the same enzyme also catalyzing the target reaction. The acetone formed when 2-propanol is used as cosubstrate can in a further preferred embodiment continuously removed from the reaction mixture.

The cofactor loading i.e. the ratio substrate (prochiral ketone) to cofactor (s/c) can vary between 10 and 3000, preferably between 50 and 1000, most preferably between 100 and 500.

In a particular embodiment of the present invention, the enzymatic reduction is performed in an aqueous buffer medium in the presence of the co-substrate i.e. preferably in the presence of 2-propanol. The concentration of the co-substrate is typically in the range of 5% v to 40% v.

Suitable buffers can be selected from acidic to neutral buffers such as 2-morpholin-4-ethanesulfonic acid, ammonium acetate, acetate, phosphate, 1,4-Piperazinediethanesulfonic acid, which allow to keep the pH of the reaction in the range between pH 5.2 and pH 7.2.

The substrate loading, i.e. the loading of the prochiral ketone may be selected between 1% wt and 20% wt, preferably between 10% wt and 20% wt and the ratio substrate to enzyme (s/e) can be selected between 25 and 200, preferably between 100 and 200.

The reaction temperature is usually kept in a range between 20° C. and 50° C., preferably between 25° C. and 45° C.

Upon termination of the reaction the resulting chiral alcohol can be conventionally worked up by extraction or preferred by filtration.

In a fifth aspect the present invention relates to the use of the enzymatic reduction of a prochiral ketone and of the formation of a chiral alcohol in the presence of mutant ketoreductase in the synthesis of morpholine compounds of formula

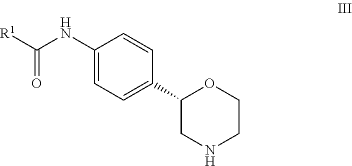

wherein R¹ is aryl or heteroaryl, wherein the aromatic rings are optionally substituted by one or two C₁₋₇-alkyl substituents.

The term “C₁₋₇-alkyl” relates to a branched or straight-chain monovalent saturated aliphatic hydrocarbon radical of one to six carbon atoms, preferably one to four, more preferably one to two carbon atoms. This term is further exemplified by radicals as methyl, ethyl, n-propyl, i-propyl, n-butyl, s-butyl or t-butyl, pentyl and its isomers, hexyl and its isomers and heptyl and its isomers.

The term “aryl”, relates to an aromatic carbon ring such as to the phenyl or naphthyl ring, preferably to the phenyl ring.

The term “heteroaryl” refers to an aromatic 5 to 6 membered monocyclic ring or 9 to 10 membered bicyclic ring which can comprise 1, 2 or 3 heteroatoms selected from nitrogen, oxygen and/or sulphur, such as pyridinyl, pyrazolyl, pyrimidinyl, benzoimidazolyl, quinolinyl and isoquinolinyl.

Preferably R¹ is heteroaryl, more preferably pyrazolyl, substituted by two C₁₋₇-alkyl, more preferably two C₁₋₂-alkyl substituents.

Even more preferably the morpholine compound is the TAARI clinical candidate ralmitaront having the formula X

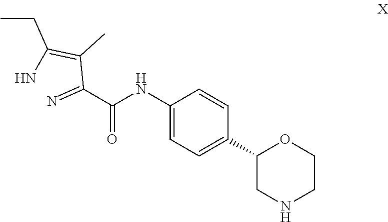 [SMILES: CC/C(N)=C(\C)C(=N)C(=O)Nc1ccc([C@H]2CNCCO2)cc1]

As described in the PCT Publication WO 2017/157873, Scheme 1, page 5, the synthesis of ralmitaront can be performed as follows:

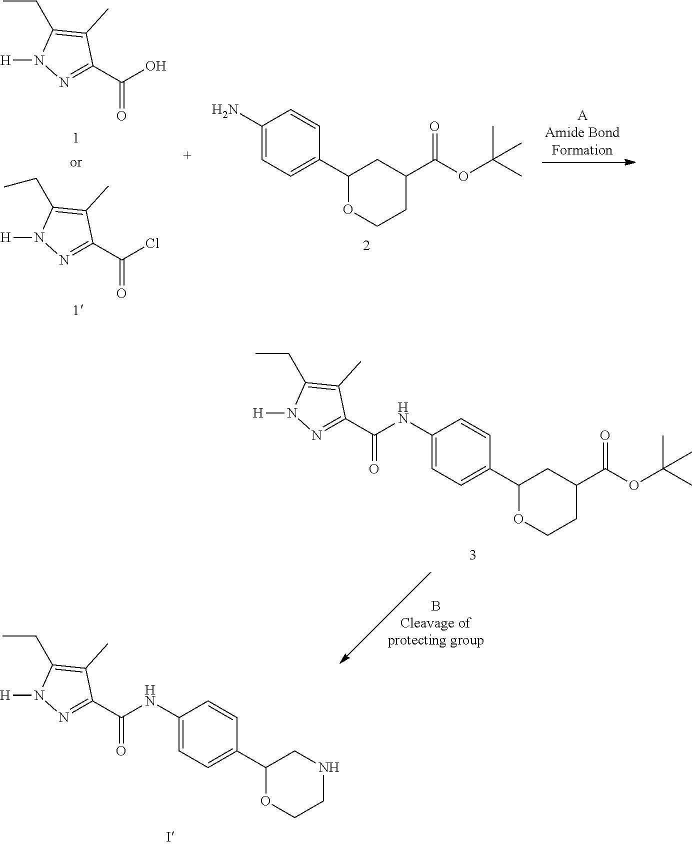

The use in accordance with the fifth aspect the present invention relates to the preparation of the 2-(4-aminophenyl) morpholine intermediate 2.

Intermediate 2 is a chiral 2-(4-aminophenyl) morpholine of the formula

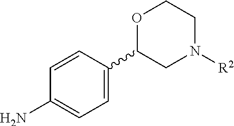

wherein R² is the Boc amino protecting group.

The preparation of intermediate 2 can be accomplished in accordance with the process as disclosed in the PCT Publication WO 2015/086495, wherein the enzymatic reduction in step a) is replaced by the enzymatic reduction of the prochiral ketone and the formation of a chiral alcohol in the presence of the mutant ketoreductase, according to the fourth aspect of the present invention. Subsequent synthesis steps b) to e) can then follow the disclosure in the PCT Publication WO 2015/086495.

With respect to the use of the present invention it is referred to the terms, examples and specific embodiments used in the context of the other aspects of the present disclosure, which are also applicable to this aspect. Particularly, the mutant ketoreductase according to the present invention or the fusion protein thereof may be used as detailed with respect to the methods of the present invention.

Unless defined otherwise, all technical and scientific terms and any acronyms used herein have the same meanings as commonly understood by one of ordinary skill in the art in the field of the invention. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The invention is not limited to the particular methodology, protocols, and reagents described herein because they may vary. Although any methods and materials similar or equivalent to those described herein can be used in the practice of the present invention, the preferred methods, and materials are described herein. Further, the terminology used herein is for the purpose of describing particular embodiments only and is not intended to limit the scope of the present invention.

As used herein and in the appended claims, the singular forms “a”, “an”, and “the” include plural reference unless the context clearly dictates otherwise. Similarly, the words “comprise”, “contain” and “encompass” are to be interpreted inclusively rather than exclusively. Similarly, the word “or” is intended to include “and” unless the context clearly indicates otherwise. The term “plurality” refers to two or more.

The following Figures and Examples are intended to illustrate various embodiments of the invention. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

### EXAMPLES

### 1 General Procedures

### 1.1 Biocatalyst Production

For ketoreductase gene acquisition and construction of expression vectors, ketoreductase (KRED) open reading frames were designed and synthesized for expression in Escherichia coli (E. coli), based on the reported amino acid sequence of the ketoreductase, and on the mutant sequences desired (provided in section Sequences), and the codon optimization algorithm of Twist Bioscience (South San Francisco, U.S.A.). A stop codon in the end was added in all cases. Restriction sites for the subsequent cloning in the vector of interest, pET-29b(+), were added in the nucleotide sequence; NdeI restriction was added in the 5′ end, and the XhoI restriction sequence in the 3′ end. The vector contains the coding sequence for kanamycin resistance (Kmr gene). According to the cloning strategy, the expression is under the control of a lac promoter. Resulting plasmids were transformed into E. coli BL21 (DE3) using standard methods. Sequences of the codon optimized genes and encoded polypeptides are provided in section “SEQUENCES”.

### 1.2 Production of Ketoreductases

Plasmids from Twist Bioscience were resuspended in sterile water. Inoculation in E. coli BL21 (DE3) cells was achieved by thermal heating (42° C. for 45 s).

A preculture was incubated overnight at 37° C., on a Luria Bertani medium agar plate, containing 25 μg/mL kanamycin. A single microbial colony was picked and incubated overnight, according to the protocol.

Terrific broth medium, containing 25 μg/mL kanamycin, was added to the culture. Following 3.5 hours incubation at 28° C., isopropyl p D-thiogalactoside (IPTG) was added at a final concentration of 1 mM to induce the expression of the KRED. Incubation continued overnight at 28° C. Cells were harvested via centrifugation (3220 rcf, 45 min, 4° C.) and the supernatant was discarded. The cells were resuspended in KPI buffer (100 mM, pH 7), 2 mM MgCl₂, 1 mg/ml lysozyme, 0.75 mg/ml polymyxin, 0.2 mg/ml DNase I and incubated for 60 min. They were then centrifuged (3220 rcf, 45 min, 4° C.) and the lysate was frozen and stored at −20° C.

### 2 Biocatalysis

### 2.1 Process Development

The enzymatic reduction takes place in a reaction mixture of a buffer (e.g.: 2-morpholin-4-ylethanesulfonic acid MES; 0.5 M stock solution used, pH 6.5) and 2-propanol (final reductant) at a defined temperature (23-45° C.). The buffer and 2-Propanol (5-40 vol %) varies in the experiments, for reaction with a substrate loading higher than 1%, the 2-PrOH concentration is at least 20%. The loading of the ketoreductase and cofactor, nicotinamide adenine dinucleotide phosphate cofactor (NADP), is defined in dependency to the substrate loading. The substrate is added in variable concentrations between 1 and 20 weight %. The enzyme loading varies between experiments, corresponding to a substrate to enzyme ratio (s/e) between 33 and 200. The cofactor loading varies between experiments, corresponding to a substrate to cofactor ratio (s/c) between 10 and 1000.

### 2.2 Analysis Methods

### Achiral HPLC Method (IPC)

The production of the alcohol is measured after 18 hours through HPLC analysis, on a C18 XP column (3.0×75 mm, 2.5 μm particle size at 50° C. and 311 bar). Phase A contains 5% Acetonitrile in water and 0.1% formic acid; Phase B contains Acetonitrile and 0.1% formic acid. Flow 1 ml/min; 90% phase A at time 0, 60% at minute 7, and 90% at minute 7.5. Detection wavelength 280 nm.

For sample preparation, the sample is diluted in acetonitrile/water 4:1 to a concentration of 1 mg/ml, for a total injection volume of 1 μl. The retention times are 4.89′ for educt; 3.65′ for product; 3.46′ for epoxid. Conversion is computed as ratio of product peak area on total area of peaks.

### Chiral HPLC Method (OP)

The enantiomeric excess of the product compound is measured by chiral analysis on a IE-3 column (4.6×150 mm, 3 μm particle size at 40° C. and 250 bar). Phase A contains 5% Acetonitrile in water; Phase B contains water, ethanol and Isopropyl alcohol in proportion 30:35:35. Flow 0.7 ml/min; 50% phase A at minute 30. Detection wavelength 264 nm.

For sample preparation, the sample is diluted in ethanol to a concentration of 1 mg/ml, for a total injection volume of 5 μl. The retention times are 8.8′ for the S product; 9.7′ for the R product; 15.8′ for epoxid and 24.8 for educt. Enantiomeric excess is computed as ratio of product R peak area minus product S peak divided by the sum of the areas of the two peaks

### 3 Mutant Design for the Improvement of the KRED Specific Activity for Ketonel

The specific activity of the presented mutants was determined according to the reaction conditions described in section 2. The positions were identified in the L. brevis R-specific alcohol dehydrogenase (Uniprot ID Q84EX5, PDB structure 1ZK4) by structural analysis. Positions in proximity of the substrate and cofactor were selected for mutations.

The wild type (WT) activity respectively conversions is reported in Table 1 and defined as Parent (under given conditions). Its Fold Improvement Over the Parent (FIOP) is 1. For the mutants, the FIOP is reported, with the wild type taken as reference (parent).

The FIOP is computed as follows:

$$FIOP = \frac{{C}_{mut}}{{C}_{WT}} \frac{{t}_{mut}}{{t}_{WT}} \frac{S / {E}_{mut}}{S / {E}_{WT}}$$

Cₘᵤₜ the conversion in area % (activity) achieved by the mutein used; C_{WT} the one of the WT-ketoreductase used
s/eₘᵤₜ the s/e (substrate to mutein ratio) for the experiment and s/e_{WT} the s/e for the experiment with the WT-ketoreductase used
reaction time (t_{WT}/tₘᵤₜ) needed to achieve Cₘᵤₜ respectively C_{WT}

The FIOP determination necessitates ideally the comparison of similar conversion degree levels, which might require different substrate concentration (c) and/or enzyme loading (S/h values) relating to the individual activity of the enzyme variants under the reaction conditions.

TABLE 1
Conversion in area % and FIOP of investigated ketoreductases
(WT and single mutants) at 1% loading and 33 S/E loading.

| Ketoreductase | Enzyme | Time | | |
|---|---|---|---|---|
| @ c = 1% | loading | (h) | conversion Area % | FIOP |
| WT | s/e 33 | 18 | 52.82 | 1.0 |
| T16S | s/e 33 | 18 | 97.08 | 1.8 |
| E145L | s/e 33 | 18 | 100* | >1.9 |

*As time to complete (100%) conversion had not been determined, the FIOP is at least the given value.

The two single mutants T16S and E145L showed a close to 2 fold improvement over the parent (here the wildtype). Mutant E145L shows complete conversion at the reaction quench, indicating that the conversion completed earlier in time.

TABLE 2
Conversion in area % and FIOP of investigated ketoreductases
(WT and single or double mutants) under different S/E loading,
at 1% substrate concentration.

| Ketoreductase | Enzyme | Time | conversion | | ee |
|---|---|---|---|---|---|
| @ c = 1% | loading | (h) | Area % | FIOP** | [%] |
| T16S | 4 μL lysate | 18 | 60 | 1.8 | 99.7 |
| E1451 | 4 μL lysate | 18 | 65 | 2.0 | |
| E145L | 4 μL lysate | 18 | 69 | 2.1 | 99.9 |
| A202I | 4 μL lysate | 18 | 74 | 2.2 | 100 |
| L199N_A202I | 4 μL lysate | 18 | 81 | 2.4 | 100 |
| E145A | 8 μL lysate | 1 | 32 | 8.6 | |
| E145C | 8 μL lysate | 1 | 32 | 8.6 | |
| E145M | 8 μL lysate | 1 | 31 | 8.4 | |
| E145T | 8 μL lysate | 1 | 30 | 8.1 | |
| T16A_E145L | 8 μL lysate | 1 | 37 | 10.0 | |
| T16C_E145L | 8 μL lysate | 1 | 31 | 8.4 | |
| T16G_E145L | 8 μL lysate | 1 | 37 | 10.0 | |
| T16I_E145L | 8 μL lysate | 1 | 31 | 8.4 | |
| T16M_E145L | 8 μL lysate | 1 | 30 | 8.2 | |
| T16S_E145L | 8 μL lysate | 1 | 32 | 8.6 | |
| T16V_E145L | 8 μL lysate | 1 | 31 | 8.4 | |
| T16Y_E145L | 8 μL lysate | 1 | 35 | 9.5 | |
| V43K_E145L | 8 μL lysate | 1 | 24 | 6.5 | |
| V43Q_E145L | 8 μL lysate | 1 | 40 | 10.8 | |
| M141I_E145L | 8 μL lysate | 1 | 45 | 12.2 | |
| I144A_E145L | 8 μL lysate | 1 | 41 | 11.1 | |
| I144C_E145L | 8 μL lysate | 1 | 35 | 9.5 | |
| I144S_E145L | 8 μL lysate | 1 | 36 | 9.7 | |
| I144T_E145L | 8 μL lysate | 1 | 38 | 10.3 | |
| I144V_E145L | 8 μL lysate | 1 | 34 | 9.2 | |
| E145L_A202C | 8 μL lysate | 1 | 46 | 12.4 | |
| E145L_A202E | 8 μL lysate | 1 | 42 | 11.3 | |
| E145L_A202I | 8 μL lysate | 1 | 47 | 12.7 | |
| E145L_A202L | 8 μL lysate | 1 | 53 | 14.3 | |
| E145L_A202T | 8 μL lysate | 1 | 49 | 13.2 | |
| L199F_A202I | 8 μL lysate | 1 | 32 | 8.6 | |
| L199M_A202I | 8 μL lysate | 1 | 27 | 7.3 | |
| L199S_A202I | 8 μL lysate | 1 | 32 | 8.6 | |
| L199Q_A202L | 8 μL lysate | 1 | 41 | 11.1 | |
| L199V_A202I | 8 μL lysate | 1 | 29 | 7.8 | |
| L199V_A202L | 8 μL lysate | 1 | 43 | 11.6 | |

**The FIOP of T16S is taken from the experiments with Lyophylisate (Table 1) - the others have been calculated based on it, according to formula 1.

Table 2 illustrates that position 16, 141, 144, 145, 199 were investigated further either as single mutants or in combination with a beneficial mutation inferred from the results of Table 1. For each position a subset of amino acids showed increased activity with respect to the parent. In particular combination of M141I, I144A, E145L, A202, A202I and A202L showed the highest conversion.

TABLE 3
Conversion in area % and FIOP of investigated ketoreductases
(WT and triple or quadruple mutants) under different S/E loading,
at 1% substrate concentration.

| | | 17 h | | |
|---|---|---|---|---|
| | Enzyme | conversion | | |
| Ketoreductase @ c = 1% | loading | Area % | FIOP | ee [%] |
| I144A_E145L_L199M_A202L | s/e 66 | 32 | 1.9 | |
| WT | s/e 33 | 33.72 | 1 | |
| I144A_E145L_L199S_A202L | s/e 66 | 28.8 | 1.7 | |
| I144A_E145L_L199M_A202L | s/e 66 | 32 | 1.9 | |
| E145L_L199N_A202L | s/e 66 | 45.3 | 2.7 | |
| I144A_E145L_L199N_A202I | s/e 66 | 48.8 | 2.9 | |
| I144A_E145L_L199M_A202I | s/e 66 | 60.9 | 3.6 | |
| E145L_L199S_A202L | s/e 66 | 63.5 | 3.8 | |
| E145L_L199M_A202L | s/e 66 | 84.4 | 5.0 | |
| E145L_L199M_A202I | s/e 66 | 100* | >5.9 | |
| E145L_L199S_A202I | s/e 66 | 100* | >5.9 | 99.9 |
| M141I_E145L_L199S_A202I | s/e 66 | 100* | >5.9 | 99.9 |
| E145L_L199N_A202I | s/e 66 | 100* | >5.9 | 100 |

*As time to complete (100%) conversion had not been determined, the FIOP is at least the given value.

Table 3 illustrates that combination of mutants from Table 2 were further investigated. Preferred mutants are reported in Table 3, and include combination of M141I, I144A, E145L, L199M/N/S, A202I/L.

TABLE 4
Conversion in area % and FIOP of investigated ketoreductases (WT,
single, triple or quadruple mutants) at 10% substrate concentration.

| | | | 17 h | | |
|---|---|---|---|---|---|
| | Enzyme | Time | conversion | | |
| Ketoreductase @ c = 10% | loading | (h) | Area % | FIOP | ee [%] |
| WT | s/e 100 | 21 | 13 | 1 | |
| E145L | s/e 100 | 21 | 46 | 3.5 | |
| E145L_L199S_A202I | s/e 100 | 4 | 19.2 | 7.7 | 100 |
| M141I_E145L_L199S_A202I | s/e 100 | 4 | 24.1 | 9.7 | 100 |
| E145L_L199N_A202I | s/e 100 | 4 | 25.1 | 10.1 | 100 |

Table 4 illustrates that effective mutants from Table 3 were successfully tested with a higher substrate concentration and high substrate to enzyme loading i.e. under technical scale conditions.

### 4 Characterization of Mutant Ketoreductases

4.1 General Screening Procedure—1% [w/v] Substrate Loading

10 mg (0.1 mmol) 2-bromo-1-(4-nitrophenyl)ethanone is mixed in a mixture of 100 μl MES-buffer pH 6.5 (0.5M), 650 μl water, 200 μl 2-propanol and 20 μl magnesium bromide hexahydrate (0.1 M). The reaction was started by the addition of 200 μg NADP and 100 μg ketoreductase at room temperature. After ˜1 d the reaction conversion had been determined by an achiral HPLC method (IPC) a chiral HPLC method (OP).

4.2 General Screening Procedure—5% [w/v] Substrate Loading

50 mg (0.2 mmol) 2-bromo-1-(4-nitrophenyl)ethanone is mixed in a mixture of 100 μl MES-buffer pH 6.5 (0.5M), 650 μl water, 355 μl 2-propanol and 20 μl magnesium bromide hexahydrate (0.1 M). The reaction was started by the addition of 1.0 mg NADP and 250 μg ketoreductase at room temperature. After 4 h and ˜21 h the reaction conversion had been determined by an achiral HPLC method (IPC) a chiral HPLC method (OP).

4.3 Temperature Stability Screening—5% [w/v] Substrate Loading

0.25 mg ketoreductase and 1.0 mg NADP were dissolved in a mixture of 0.1 ml MES-buffer pH 6.5 (0.5M), 355 μl water and 20 μl magnesium bromide hexahydrate (0.1 M) at different temperatures ranging from 25° C. to 45° C. (see Table 5). After 16.5 h incubation under shaking (1000 rpm) the reactions were started by the addition of 50 mg (0.2 mmol) 2-bromo-1-(4-nitrophenyl)ethanone dissolved in 0.4 ml 2-propanol. After 4 h and 23 h the reaction conversion had been determined by an achiral HPLC method (IPC). All results are summarized in Table 5.

TABLE 5
Temperature stability screening of investigated ketoreductases.

| | | product | product |
|---|---|---|---|
| | Temperature | [a %] | [a %] |
| Ketoreductase | [° C.] | 4 h | 23 h |
| E145L_L199N_A202I | 25 | 21.1 | 98.1 |
| E145L_L199N_A202I | 30 | 22.3 | 97.4 |
| E145L_L199N_A202I | 35 | 9.6 | 87.4 |
| E145L_L199N_A202I | 40 | 0.8 | 15.1 |
| E145L_L199N_A202I | 45 | 0.4 | 5.2 |
| E145L_L199S_A202I | 25 | 6.2 | 98.0 |
| E145L_L199S_A202I | 30 | 13.6 | 98.5 |
| E145L_L199S_A202I | 35 | 4.7 | 83.2 |
| E145L_L199S_A202I | 40 | 1.8 | 30.4 |
| E145L_L199S_A202I | 45 | 0.5 | 5.3 |
| M141I_E145L_L199S_A202I | 25 | 24.3 | 98.6 |
| M141I_E145L_L199S_A202I | 30 | 13.8 | 98.0 |
| M141I_E145L_L199S_A202I | 35 | 10.1 | 98.6 |
| M141I_E145L_L199S_A202I | 40 | 2.0 | 21.1 |
| M141I_E145L_L199S_A202I | 45 | 0.4 | 4.7 |

Table 5 illustrates that the three selected mutants show similar performances at the 23 hour time point for a temperature of 25 and 30° C., despite different initial activities (4 hour time point). The quadruple mutant show best performance at 35° C.

4.4 Up-Scaled Production—10% [w/v] Substrate Loading

3 g (12.3 mmol) 2-bromo-1-(4-nitrophenyl)ethanone is suspended under stirring in a mixture of 3 ml MES-buffer pH 6.5 (0.5M), 11.4 ml water, 12 ml 2-propanol and 0.6 ml magnesium bromide hexahydrate (0.1 M). The reaction was started by the addition of 30 mg NADP and 15 mg ketoreductase at room temperature. At reaction completion the final pH was 6.1. After 4 h and 21 h the reaction conversion had been determined by an achiral HPLC method (IPC) and the enantiomeric excess after 21 h by a chiral HPLC method (OP). All results are summarized in Table 6.

TABLE 6
Characterization of investigated ketoreductases at 10% [w/v] substrate
loading in up-scaled process.

| | Product | Product | Chiral |
|---|---|---|---|
| | [a %] | [a %] | HPLC |
| Ketoreductase | 4 h | 21 h | 21 h |
| E145L_L199S_A202I | 31.8 | 100 | 100% (S) |
| E145L_L199N_A202I | 34.7 | 100 | 100% (S) |
| M141I_E145L_L199S_A202I | 40.2 | 100 | 100% (S) |

Table 6 illustrates that the three selected mutants show complete conversion after 21 hours in experiments at larger scale.

4.5 Up-Scaled Production—20% [w/v] Substrate Loading

6 g (24.6 mmol) 2-bromo-1-(4-nitrophenyl)ethanone is suspended under stirring in a mixture of 3 ml MES-buffer pH 6.5 (0.5M), 8.4 ml water, 12 ml 2-propanol (final reductant) and 0.6 ml magnesium bromide hexahydrate (0.1 M). The reaction was started by the addition of 60 mg NADP and 30 mg ketoreductase (see Table 7) at room temperature. At reaction completion the final pH was 5.8. After 4 h, 21 h and 2 d the reaction conversion had been determined by an achiral HPLC method (IPC) and the enantiomeric excess after 2 d by a chiral HPLC method (OP). All results are summarized in Table 7.

TABLE 7
Characterization of investigated ketoreductases at 20% [w/v] substrate
loading In up-scaled process.

| | Product | Product | Product | Chiral | |
|---|---|---|---|---|---|
| | [a %] | [a %] | [a %] | HPLC | |
| Ketoreductase | 4 h | 1 d | 2 d | 2 d | |
| E145L_L199S_A202I | 19.2/15.5 | 62.7/53.8 | 98.6/84.2 | >99.9 | (S) |
| E145L_L199N_A202I | 24.1/25.9 | 78.1/83.0 | 98.8/99.1 | 100 | (S) |
| M141I_E145L_L199S_ | 25.1/33.2 | 78.0/100 | 98.8/100 | >99.9 | (S) |
| A202I | | | | | |
| M141I_E145L_L199N_ | 23.7 | 70.6 | 99.8 | >99.9 | (S) |
| A202I | | | | | |

Table 7 shows that complete conversion and excellent enantiomeric excess of the product can be achieved also at 20% substrate loading within 2 days.

SEQUENCES
Amino acids sequences
>Lactobacillus brevis ATCC 14869 ketoreductase, referred to as Q84EX5 in
UniProtKB
(SEQ ID NO: 1)
MSNRLDGKVAIITGGTLGIGLAIATKFVEEGAKVMITGRHSDVGEKAAKSVGTPDQIQFFQ
HDSSDEDGWTKLFDATEKAFGPVSTLVNNAGIAVNKSVEETTTAEWRKLLAVNLDGVFFGT
RLGIQRMKNKGLGASIINMSSIEGFVGDPSLGAYNASKGAVRIMSKSAALDCALKDYDVRV
NTVHPGYIKTPLVDDLPGAEEAMSQRTKTPMGHIGEPNDIAYICVYLASNESKFATGSEFVVD
GGYTAQ
>Q84EX5_E145L_A202L
(SEQ ID NO: 2)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDLPGLEEAMSQRTKTPMGHIGEPNDIAYICVYLASNESKFATGSEFVVDGGYTAQ
>Q84EX5_E145L_A202C
(SEQ ID NO: 3)
MSNRLDGKVAIITGGTLGIGLAIATKFVEEGAKVMITGRHSDQGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDLPGCEEAMSQRTKTPMGHIGEPNDIAYICVYLASNESKFATGSEFVVDGGYTAQ
>Q84EX5_E145L_A202E
(SEQ ID NO: 4)
MSNRLDGKVAIITGGTLGIGLAIATKFVEEGAKVMITGRHSDQGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDLPGEEEAMSQRTKTPMGHIGEPNDIAYICVYLASNESKFATGSEFVVDGGYTAQ
>Q84EX5_E145L_A202I
(SEQ ID NO: 5)
MSNRLDGKVAIITGGTLGIGLAIATKFVEEGAKVMITGRHSDQGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDLPGIEEAMSQRTKTPMGHIGEPNDIAYICVYLASNESKFATGSEFVVDGGYTAQ
>Q84EX5_E145L A202T
(SEQ ID NO: 6)
MSNRLDGKVAIITGGTLGIGLAIATKFVEEGAKVMITGRHSDQGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDLPGTEEAMSQRTKTPMGHIGEPNDIAYICVYLASNESKFATGSEFVVDGGYTAQ
>Q84EX5_E145L_L199M_A202I
(SEQ ID NO: 7)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDMPGIEEAMSQRTKTPMGHIGEPNDIAYICVYLASNESKFATGSEFVVDGGYTAQ
>Q84EX5_E145L_L199M_A202L
(SEQ ID NO: 8)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDMPGLEEAMSQRTKTPMGHIGEPNDIAYICVYLASNESKFATGSEFVVDGGYTAQ
>Q84EX5_E145L_L199N_A202L
(SEQ ID NO: 9)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDNPGLEEAMSQRTKTPMGHIGEPNDIAYICVYLASNESKFATGSEFVVDGGYTAQ
>Q84EX5_E145L_L199N_A202I
(SEQ ID NO: 10)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDNPGIEEAMSQRTKTPMGHIGEPNDIAYICVYLASNESKFATGSEFVVDGGYTAQ
>Q84EX5_E145L_L199S_A202L
(SEQ ID NO: 11)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDSPGLEEAMSQRTKTPMGHIGEPNDIAYICVYLASNESKFATGSEFVVDGGYTAQ
>Q84EX5_E145L_L199S_A202I
(SEQ ID NO: 12)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSILGFVGDPSLGAYNASKGAVRIMSKSAALDCALKDYDVRVNTVHPG
YIKTPLVDDSPGIEEAMSQRTKTPMGHIGEPNDIAYICVYLASNESKFATGSEFVVDGGYTAQ
>Q84EX5_M141I_E145L_L199S_A202I
(SEQ ID NO: 13)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINISSILGFVGDPSLGAYNASKGAVRIMSKSAALDCALKDYDVRVNTVHPG
YIKTPLVDDSPGIEEAMSQRTKTPMGHIGEPNDIAYICVYLASNESKFATGSEFVVDGGYTAQ
>Q84EX5_I144A_E145L_L199M_A202I
(SEQ ID NO: 14)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSALGFVGDPSLGAYNASKGAVRIMSKSAALDCALKDYDVRVNTVHPG
YIKTPLVDDMPGIEEAMSQRTKTPMGHIGEPNDIAYICVYLASNESKFATGSEFVVDGGYTAQ
>Q84EX5_I144A_E145L_L199N_A202I
(SEQ ID NO: 15)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSALGFVGDPSLGAYNASKGAVRIMSKSAALDCALKDYDVRVNTVHPG
YIKTPLVDDNPGIEEAMSQRTKTPMGHIGEPNDIAYICVYLASNESKFATGSEFVVDGGYTAQ
>Q84EX5_I144A_E145L_L199M_A202L
(SEQ ID NO: 16)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSALGFVGDPSLGAYNASKGAVRIMSKSAALDCALKDYDVRVNTVHPG
YIKTPLVDDMPGLEEAMSQRTKTPMGHIGEPNDIAYICVYLASNESKFATGSEFVVDGGYTAQ
>Q84EX5_I144A_E145L_L199S_A202L
(SEQ ID NO: 17)
MSNRLDGKVAIITGGTLGIGLAIATKFVEEGAKVMITGRHSDVGEKAAKSVGTPDQIQFFQHD
SSDEDGWTKLFDATEKAFGPVSTLVNNAGIAVNKSVEETTTAEWRKLLAVNLDGVFFGTRLGI
QRMKNKGLGASIINMSSALGFVGDPSLGAYNASKGAVRIMSKSAALDCALKDYDVRVNTVHPG
YIKTPLVDDSPGLEEAMSQRTKTPMGHIGEPNDIAYICVYLASNESKFATGSEFVVDGGYTAQ
Nucleic acids sequences
>Lactobacillus brevis ATCC 14869 ketoreductase, referred to as Q84EX5 in
UniProtKB
(SEQ ID NO: 18)
ATGAGCAACCGTCTGGACGGCAAGGTGGCGATCATTACCGGTGGCACCCTGGGTATTGGTCTG
GCGATTGCGACCAAGTTCGTGGAGGAAGGTGCGAAAGTTATGATCACCGGCCGTCACAGCGAC
GTGGGCGAGAAGGCGGCGAAAAGCGTTGGCACCCCGGACCAGATTCAATTCTTTCAGCACGAT
AGCAGCGACGAGGATGGTTGGACCAAGCTGTTCGATGCGACCGAAAAAGCGTTTGGCCCGGTT
AGCACCCTGGTTAACAACGCGGGTATTGCGGTGAACAAGAGCGTTGAGGAAACCACCACCGCG
GAGTGGCGTAAACTGCTGGCGGTGAACCTGGATGGTGTTTTCTTTGGCACCCGTCTGGGTATC
CAACGTATGAAGAACAAAGGTCTGGGCGCGAGCATCATTAACATGAGCAGCATTGAAGGTTTC
GTTGGCGACCCGAGCCTGGGTGCGTACAACGCGAGCAAGGGTGCGGTTCGTATCATGAGCAAA
AGCGCGGCGCTGGATTGCGCGCTGAAGGACTACGATGTGCGTGTTAACACCGTGCACCCGGGC
TATATTAAAACCCCGCTGGTTGACGATCTGCCGGGTGCGGAGGAAGCGATGAGCCAGCGTACC
AAGACCCCGATGGGTCACATCGGCGAACCGAACGACATCGCGTACATTTGCGTTTATCTGGCG
AGCAACGAGAGCAAATTCGCGACCGGTAGCGAATTTGTGGTTGATGGTGGCTATACCGCGCAA
TAA
>Q84EX5_E145L_A202L
(SEQ ID NO: 19)
ATGTCCAATCGCTTGGACGGGAAGGTTGCGATTATTACCGGTGGCACCCTGGGCATCGGCCTG
GCGATCGCTACTAAATTTGTGGAAGAAGGTGCCAAGGTCATGATTACCGGCCGTCACAGCGAT
GTAGGCGAAAAAGCAGCAAAGTCCGTCGGGACCCCTGATCAGATTCAATTCTTTCAACACGAT
TCGAGCGACGAGGATGGATGGACTAAATTGTTTGATGCCACCGAAAAGGCATTCGGTCCTGTA
AGTACCTTGGTCAACAATGCAGGCATCGCTGTAAACAAAAGCGTCGAGGAGACTACTACGGCA
GAATGGCGCAAACTTCTGGCCGTCAACTTGGACGGCGTTTTTTTTGGCACGCGTCTGGGCATT
CAACGTATGAAAAACAAAGGTTTGGGAGCGTCCATCATCAATATGAGCAGCATCCTTGGATTC
GTAGGGGACCCGTCGCTGGGTGCATACAACGCCTCGAAAGGGGCGGTGCGCATTATGTCAAAA
AGCGCGGCCCTGGACTGTGCCTTAAAAGATTATGATGTACGCGTGAACACAGTTCATCCCGGT
TACATTAAAACCCCGCTTGTCGATGATCTCCCCGGCCTGGAGGAAGCGATGTCTCAGCGCACC
AAAACGCCGATGGGCCACATTGGCGAACCTAACGATATCGCATATATTTGCGTTTACCTGGCA
AGCAATGAATCTAAATTTGCGACCGGCTCAGAGTTTGTGGTGGATGGCGGCTATACCGCGCAG
TAA
>Q84EX5_E145L_A202C
(SEQ ID NO: 20)
ATGTCCAATCGCTTGGACGGGAAGGTTGCGATTATTACCGGTGGCACCCTGGGCATCGGCCTG
GCGATCGCTACTAAATTTGTGGAAGAAGGTGCCAAGGTCATGATTACCGGCCGTCACAGCGAT
GTAGGCGAAAAAGCAGCAAAGTCCGTCGGGACCCCTGATCAGATTCAATTCTTTCAACACGAT
TCGAGCGACGAGGATGGATGGACTAAATTGTTTGATGCCACCGAAAAGGCATTCGGTCCTGTA
AGTACCTTGGTCAACAATGCAGGCATCGCTGTAAACAAAAGCGTCGAGGAGACTACTACGGCA
GAATGGCGCAAACTTCTGGCCGTCAACTTGGACGGCGTTTTTTTTGGCACGCGTCTGGGCATT
CAACGTATGAAAAACAAAGGTTTGGGAGCGTCCATCATCAATATGAGCAGCATCCTTGGATTC
GTAGGGGACCCGTCGCTGGGTGCATACAACGCCTCGAAAGGGGCGGTGCGCATTATGTCAAAA
AGCGCGGCCCTGGACTGTGCCTTAAAAGATTATGATGTACGCGTGAACACAGTTCATCCCGGT
TACATTAAAACCCCGCTTGTCGATGATCTCCCCGGCTGCGAGGAAGCGATGTCTCAGCGCACC
AAAACGCCGATGGGCCACATTGGCGAACCTAACGATATCGCATATATTTGCGTTTACCTGGCA
AGCAATGAATCTAAATTTGCGACCGGCTCAGAGTTTGTGGTGGATGGCGGCTATACCGCGCAG
TAA
>Q84EX5_E145L_A202E
(SEQ ID NO: 21)
ATGTCCAATCGCTTGGACGGGAAGGTTGCGATTATTACCGGTGGCACCCTGGGCATCGGCCTG
GCGATCGCTACTAAATTTGTGGAAGAAGGTGCCAAGGTCATGATTACCGGCCGTCACAGCGAT
GTAGGCGAAAAAGCAGCAAAGTCCGTCGGGACCCCTGATCAGATTCAATTCTTTCAACACGAT
TCGAGCGACGAGGATGGATGGACTAAATTGTTTGATGCCACCGAAAAGGCATTCGGTCCTGTA
AGTACCTTGGTCAACAATGCAGGCATCGCTGTAAACAAAAGCGTCGAGGAGACTACTACGGCA
GAATGGCGCAAACTTCTGGCCGTCAACTTGGACGGCGTTTTTTTTGGCACGCGTCTGGGCATT
CAACGTATGAAAAACAAAGGTTTGGGAGCGTCCATCATCAATATGAGCAGCATCCTTGGATTC
GTAGGGGACCCGTCGCTGGGTGCATACAACGCCTCGAAAGGGGCGGTGCGCATTATGTCAAAA
AGCGCGGCCCTGGACTGTGCCTTAAAAGATTATGATGTACGCGTGAACACAGTTCATCCCGGT
TACATTAAAACCCCGCTTGTCGATGATCTCCCCGGCGAAGAGGAAGCGATGTCTCAGCGCACC
AAAACGCCGATGGGCCACATTGGCGAACCTAACGATATCGCATATATTTGCGTTTACCTGGCA
AGCAATGAATCTAAATTTGCGACCGGCTCAGAGTTTGTGGTGGATGGCGGCTATACCGCGCAG
TAA
>Q84EX5_E145L_A2021
(SEQ ID NO: 22)
ATGTCCAATCGCTTGGACGGGAAGGTTGCGATTATTACCGGTGGCACCCTGGGCATCGGCCTG
GCGATCGCTACTAAATTTGTGGAAGAAGGTGCCAAGGTCATGATTACCGGCCGTCACAGCGAT
GTAGGCGAAAAAGCAGCAAAGTCCGTCGGGACCCCTGATCAGATTCAATTCTTTCAACACGAT
TCGAGCGACGAGGATGGATGGACTAAATTGTTTGATGCCACCGAAAAGGCATTCGGTCCTGTA
AGTACCTTGGTCAACAATGCAGGCATCGCTGTAAACAAAAGCGTCGAGGAGACTACTACGGCA
GAATGGCGCAAACTTCTGGCCGTCAACTTGGACGGCGTTTTTTTTGGCACGCGTCTGGGCATT
CAACGTATGAAAAACAAAGGTTTGGGAGCGTCCATCATCAATATGAGCAGCATCCTTGGATTC
GTAGGGGACCCGTCGCTGGGTGCATACAACGCCTCGAAAGGGGCGGTGCGCATTATGTCAAAA
AGCGCGGCCCTGGACTGTGCCTTAAAAGATTATGATGTACGCGTGAACACAGTTCATCCCGGT
TACATTAAAACCCCGCTTGTCGATGATCTCCCCGGCATTGAGGAAGCGATGTCTCAGCGCACC
AAAACGCCGATGGGCCACATTGGCGAACCTAACGATATCGCATATATTTGCGTTTACCTGGCA
AGCAATGAATCTAAATTTGCGACCGGCTCAGAGTTTGTGGTGGATGGCGGCTATACCGCGCAG
TAA
>Q84EX5_E145L_A202T
(SEQ ID NO: 23)
ATGTCCAATCGCTTGGACGGGAAGGTTGCGATTATTACCGGTGGCACCCTGGGCATCGGCCTG
GCGATCGCTACTAAATTTGTGGAAGAAGGTGCCAAGGTCATGATTACCGGCCGTCACAGCGAT
GTAGGCGAAAAAGCAGCAAAGTCCGTCGGGACCCCTGATCAGATTCAATTCTTTCAACACGAT
TCGAGCGACGAGGATGGATGGACTAAATTGTTTGATGCCACCGAAAAGGCATTCGGTCCTGTA
AGTACCTTGGTCAACAATGCAGGCATCGCTGTAAACAAAAGCGTCGAGGAGACTACTACGGCA
GAATGGCGCAAACTTCTGGCCGTCAACTTGGACGGCGTTTTTTTTGGCACGCGTCTGGGCATT
CAACGTATGAAAAACAAAGGTTTGGGAGCGTCCATCATCAATATGAGCAGCATCCTTGGATTC
GTAGGGGACCCGTCGCTGGGTGCATACAACGCCTCGAAAGGGGCGGTGCGCATTATGTCAAAA
AGCGCGGCCCTGGACTGTGCCTTAAAAGATTATGATGTACGCGTGAACACAGTTCATCCCGGT
TACATTAAAACCCCGCTTGTCGATGATCTCCCCGGCACCGAGGAAGCGATGTCTCAGCGCACC
AAAACGCCGATGGGCCACATTGGCGAACCTAACGATATCGCATATATTTGCGTTTACCTGGCA
AGCAATGAATCTAAATTTGCGACCGGCTCAGAGTTTGTGGTGGATGGCGGCTATACCGCGCAG
TAA
>Q84EX5_E145L_L199M_A202I
(SEQ ID NO: 24)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCATTCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATATGCCGGGCATTGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_E145L_L199M_A202L
(SEQ ID NO: 25)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCATTCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATATGCCGGGCCTGGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_E145L_L199N_A202L
(SEQ ID NO: 26)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCATTCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAACCCGGGCCTGGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_E145L_L199N_A202I
(SEQ ID NO: 27)
ATGAGCAATCGTCTGGATGGAAAGGTAGCAATTATTACCGGCGGGACTCTGGGCATTGGACTC
GCGATTGCGACAAAATTCGTGGAAGAAGGCGCGAAAGTGATGATTACGGGTCGCCATTCGGAC
GTAGGGGAAAAAGCTGCGAAAAGTGTTGGCACTCCGGACCAGATTCAGTTTTTTCAACATGAT
TCCTCCGATGAGGATGGCTGGACGAAATTATTCGACGCGACCGAAAAAGCATTTGGGCCGGTC
TCAACATTGGTCAATAATGCTGGCATCGCCGTCAATAAATCTGTCGAAGAAACCACCACCGCT
GAATGGCGCAAACTGCTGGCCGTCAATCTGGATGGCGTTTTCTTTGGTACGCGGCTCGGGATT
CAGCGGATGAAGAACAAAGGGCTGGGGGCAAGTATCATTAATATGTCGAGCATCCTTGGGTTT
GTCGGCGACCCCTCATTAGGGGCCTACAACGCTAGCAAAGGTGCCGTACGCATCATGAGCAAA
TCTGCGGCGTTGGACTGCGCCCTGAAAGATTACGATGTGCGCGTTAATACCGTCCATCCGGGT
TATATTAAAACGCCGTTGGTAGATGATAACCCAGGTATCGAGGAAGCAATGTCCCAGCGCACC
AAAACCCCAATGGGACATATTGGCGAACCGAACGATATTGCCTATATTTGTGTATACCTGGCG
TCAAATGAGTCTAAATTTGCGACGGGGAGCGAATTTGTGGTAGATGGCGGCTACACCGCGCAA
TAA
>Q84EX5_E145L_L199S_A202L
(SEQ ID NO: 28)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCATTCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAGCCCGGGCCTGGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_E145L_L199S_A202I
(SEQ ID NO: 29)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCATTGCGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAGCCCGGGCATTGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_M141I_E145L_L199S_A202I
(SEQ ID NO: 30)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATTAGCAGCATTCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAGCCCGGGCATTGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_I144A_E145L_L199M_A202I
(SEQ ID NO: 31)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCGCGCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATATGCCGGGCATTGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_I144A_E145L_L199N_A202I
(SEQ ID NO: 32)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCGCGCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAACCCGGGCATTGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_I144A_E145L_L199M_A202L
(SEQ ID NO: 33)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCGCGCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATATGCCGGGCCTGGAAGAAGCGATGAGCCAGCGCACC
AAAACCCCGATGGGCCATATTGGCGAACCGAACGATATTGCGTATATTTGCGTGTATCTGGCG
AGCAACGAAAGCAAATTTGCGACCGGCAGCGAATTTGTGGTGGATGGCGGCTATACCGCGCAG
TAA
>Q84EX5_I144A_E145L_L199S_A202L
(SEQ ID NO: 34)
ATGAGCAACCGCCTGGATGGCAAAGTGGCGATTATTACCGGCGGCACCCTGGGCATTGGCCTG
GCGATTGCGACCAAATTTGTGGAAGAAGGCGCGAAAGTGATGATTACCGGCCGCCATAGCGAT
GTGGGCGAAAAAGCGGCGAAAAGCGTGGGCACCCCGGATCAGATTCAGTTTTTTCAGCATGAT
AGCAGCGATGAAGATGGCTGGACCAAACTGTTTGATGCGACCGAAAAAGCGTTTGGCCCGGTG
AGCACCCTGGTGAACAACGCGGGCATTGCGGTGAACAAAAGCGTGGAAGAAACCACCACCGCG
GAATGGCGCAAACTGCTGGCGGTGAACCTGGATGGCGTGTTTTTTGGCACCCGCCTGGGCATT
CAGCGCATGAAAAACAAAGGCCTGGGCGCGAGCATTATTAACATGAGCAGCGCGCTGGGCTTT
GTGGGCGATCCAAGCTTGGGCGCGTATAACGCGAGCAAAGGCGCGGTGCGCATTATGAGCAAA
AGCGCGGCGCTGGATTGCGCGCTGAAAGATTATGATGTGCGCGTGAACACCGTGCATCCGGGC
TATATTAAAACCCCGCTGGTGGATGATAGCCCGGGCCTGGAAGAAGCGATGAGCCAGCGCACC
AAAACCCCGATGGGCCATATTGGCGAACCGAACGATATTGCGTATATTTGCGTGTATCIGGCG
AGCAACGAAAGCAAATTTGCGACCGGCAGCGAATTTGTGGTGGATGGCGGCTATACCGCGCAG
TAA

1. A mutant ketoreductase with increased ketoreductase activity relative to the wild-type ketoreductase,
wherein the mutant ketoreductase comprises an amino acid sequence that is at least 80% identical to the amino acid sequence of SEQ ID NO: 1 (Lactobacillus brevis ATCC 14869 ketoreductase); and
wherein the mutant ketoreductase has at least two amino acid substitutions relative to the amino acid sequence of SEQ ID NO: 1, wherein
the amino acid at the position corresponding to position 145 of SEQ ID NO: 1 is substituted with Leu, Ala, Cys, Met or Thr (Leu145, Ala145, Cys145, Met145 or Thr145, respectively) and
the amino acid at the position corresponding to position 202 of SEQ ID NO: 1 is substituted with Cys, Glu, Ile, Leu or Thr (Cys202, Glu202, Ile202, Leu202 or Thr202, respectively).

2. The mutant ketoreductase of claim 1, wherein
the amino acid at the position corresponding to position 145 of SEQ ID NO: 1 is substituted with Leu (Leu145) and/or
the amino acid at the position corresponding to position 202 of SEQ ID NO: 1 is substituted with Ile (Ile202) or Leu (Leu202).

3. The mutant ketoreductase of claim 1, wherein
the amino acid at the position corresponding to position 16 of SEQ ID NO: 1 is substituted with Ala, Cys, Gly, Ile, Met, Ser, Tyr or Val (Ala16, Cys16, Gly16, Ile16, Met16, Ser16, Tyr16 or Val16, respectively); and/or
the amino acid at the position corresponding to position 43 of SEQ ID NO: 1 is substituted with Gln or Lys (Gln43 or Lys43); and/or
the amino acid at the position corresponding to position 141 of SEQ ID NO: 1 is substituted with Ile (Ile141); and/or
the amino acid at the position corresponding to position 144 of SEQ ID NO: 1 is substituted with Ala, Cys, Ser, Thr or Val (Ala144, Cys144, Ser144, Thr144 or Val144, respectively); and/or
the amino acid at the position corresponding to position 199 of SEQ ID NO: 1 is substituted with Asn, Phe, Met, Gln, Ser or Val (Asn199, Phe199, Met199, Gln199, Ser199 or Val199, respectively).

4. The mutant ketoreductase of claim 1, wherein
the amino acid at the position corresponding to position 145 of SEQ ID NO: 1 is substituted with Leu (Leu145) and
the amino acid at the position corresponding to position 199 of SEQ ID NO: 1 is substituted with Asn (Asn199) and
the amino acid at the position corresponding to position 202 of SEQ ID NO: 1 is substituted with Ile (Ile202);
or
the amino acid at the position corresponding to position 145 of SEQ ID NO: 1 is substituted with Leu (Leu145) and
the amino acid at the position corresponding to position 199 of SEQ ID NO: 1 is substituted with Ser (Ser199) and
the amino acid at the position corresponding to position 202 of SEQ ID NO: 1 is substituted with Ile (Ile202).

5. The mutant ketoreductase of claim 1, wherein
the amino acid at the position corresponding to position 141 of SEQ ID NO: 1 is substituted with Ile (Ile141) and
the amino acid at the position corresponding to position 145 of SEQ ID NO: 1 is substituted with Leu (Leu145) and
the amino acid at the position corresponding to position 199 of SEQ ID NO: 1 is substituted with Asn (Asn199) and
the amino acid at the position corresponding to position 202 of SEQ ID NO: 1 is substituted with Ile (Ile202);
or
the amino acid at the position corresponding to position 141 of SEQ ID NO: 1 is substituted with Ile (Ile141) and
the amino acid at the position corresponding to position 145 of SEQ ID NO: 1 is substituted with Leu (Leu145) and
the amino acid at the position corresponding to position 199 of SEQ ID NO: 1 is substituted with Ser (Ser199) and
the amino acid at the position corresponding to position 202 of SEQ ID NO: 1 is substituted with Ile (Ile202).

6. The mutant ketoreductase of claim 1, wherein the mutant ketoreductase does not comprise a mutation at one or more of positions corresponding to positions 94, 96, 153, 190, 195, 206 and 233 of SEQ ID NO: 1.

7. The mutant ketoreductase of claim 1, wherein the mutant ketoreductase consists of or comprises an amino acid sequence that is at least 85%, 90%, 95%, 96%, 97%, 98%, 99%, or 100% identical to the amino acid sequence of any of SEQ ID NO: 2 to 14.

8. The mutant ketoreductase of claim 1, wherein the ketoreductase activity relative to the wild-type ketoreductase is increased by at least 2.0, 5.0, or 10-fold.

9. The mutant ketoreductase of claim 1, wherein the mutant ketoreductase has an increased conversion relative to the wild-type ketoreductase at 10% substrate loading and at a mutant or wild-type ketoreductase loading of 1% [w/w] (s/e 100) using 2-Propanol recycling system.

10. The mutant ketoreductase of claim 1, wherein the mutant ketoreductase is capable of converting a prochiral ketone into a chiral alcohol.

11. The mutant ketoreductase of claim 10, wherein the prochiral ketone has the formula II 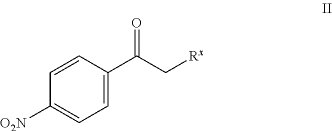
wherein R^{x} is hydrogen, C₁₋₄ alkyl or a halogen atom and the resulting chiral alcohol has the formula I 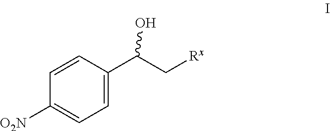
wherein R^{x} is hydrogen, C₁₋₄ alkyl or a halogen atom.

12. The mutant ketoreductase of claim 11, wherein the mutant ketoreductase has the potential to convert the ketone of formula (II) into the S-enatiomer of the chiral alcohol of formula (I) with an enantiomeric excess of at least 95%, 96%, 97%, 98% or 99%.

13. A nucleic acid coding for the mutant ketoreductase of claim 1, optionally comprised in a vector.

14. A method for the enzymatic reduction of a prochiral ketone and the formation of a chiral alcohol in the presence of mutant ketoreductase of claim 1.

15. The method of claim 14, wherein the prochiral ketone has the formula II 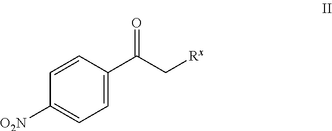
wherein R^{x} is hydrogen, C₁₋₄ alkyl or a halogen atom and the resulting chiral alcohol has the formula I 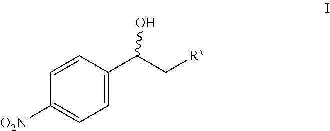
wherein R^{x} is hydrogen, C₁₋₄ alkyl or a halogen atom.

16. The method of claim 15, wherein the resulting chiral alcohol is the S-enantiomer.

17. The method of claim 14, further comprising preparing morpholine compounds of formula III 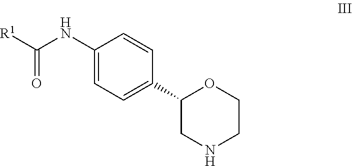
wherein R¹ is aryl or heteroaryl, wherein the aromatic rings are optionally substituted by one or two C₁₋₇-alkyl substituents.

18. The method of claim 17, wherein R¹ is pyrazolyl, substituted by two C₁₋₇-alkyl substituents.

19. The method of claim 17, wherein the morpholine compound is ralmitaront having the formula X 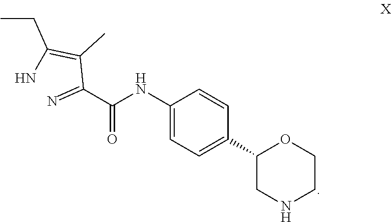 [SMILES: CC/C(N)=C(\C)C(=N)C(=O)Nc1ccc([C@H]2CNCCO2)cc1]
